SEQUENCE LISTING (14 sequences)

SEQ ID NO: 1 - protein, 10 aa, Unknown
  reference peptide used for detection in mass spectroscopy
LLDSLPSDTR

SEQ ID NO: 2 - protein, 14 aa, Unknown
  reference peptide used for detection in mass spectroscopy
SSGLVSNAPGVQIR

SEQ ID NO: 3 - protein, 11 aa, Unknown
  reference peptide used for detection in mass spectroscopy
EGYYGYTGAFR

SEQ ID NO: 4 - protein, 8 aa, Unknown
  reference peptide used for detection in mass spectroscopy
ILDDLSPR

SEQ ID NO: 5 - protein, 12 aa, Unknown
  reference peptide used for detection in mass spectroscopy
STVLTIPEIIIK

SEQ ID NO: 6 - protein, 10 aa, Unknown
  reference peptide used for detection in mass spectroscopy
TGYYFDGISR

SEQ ID NO: 7 - protein, 12 aa, Unknown
  reference peptide used for detection in mass spectroscopy
AAISGENAGLVR

SEQ ID NO: 8 - protein, 10 aa, Unknown
  reference peptide used for detection in mass spectroscopy
  MISC_FEATURE (1)..(10): some atoms of each amino acid may be heavy isotope versions
LLDSLPSDTR

SEQ ID NO: 9 - protein, 14 aa, Unknown
  reference peptide used for detection in mass spectroscopy
  MISC_FEATURE (1)..(14): some atoms of each amino acid may be heavy isotope versions
SSGLVSNAPGVQIR

SEQ ID NO: 10 - protein, 11 aa, Unknown
  reference peptide used for detection in mass spectroscopy
  MISC_FEATURE (1)..(11): some atoms of each amino acid may be heavy isotope versions
EGYYGYTGAFR

SEQ ID NO: 11 - protein, 8 aa, Unknown
  reference peptide used for detection in mass spectroscopy
  MISC_FEATURE (1)..(11): some atoms of each amino acid may be heavy isotope versions
ILDDLSPR

SEQ ID NO: 12 - protein, 12 aa, Unknown
  reference peptide used for detection in mass spectroscopy
  MISC_FEATURE (1)..(12): some atoms of each amino acid may be heavy isotope versions
STVLTIPEIIIK

SEQ ID NO: 13 - protein, 10 aa, Unknown
  reference peptide used for detection in mass spectroscopy
  MISC_FEATURE (1)..(10): some atoms of each amino acid may be heavy isotope versions
TGYYFDGISR

SEQ ID NO: 14 - protein, 17 aa, Unknown
  reference peptide used for detection in mass spectroscopy
  MISC_FEATURE (1)..(17): some atoms of each amino acid may be heavy isotope versions
AAISGENAGLVISTPER

(19) United States
(12) Patent Application Publication
(10) Pub. No.: US 2021/0057039 A1
(43) Pub. Date: Feb. 25, 2021

(54) SYSTEMS AND METHODS OF USING MACHINE LEARNING ANALYSIS TO STRATIFY RISK OF SPONTANEOUS PRETERM BIRTH

(71) Applicant: NX Prenatal Inc., Louisville, KY (US)

(72) Inventors: Brian D. BROHMAN, Louisville, KY (US); Zhen ZHANG, Dayton, MD (US); Robert C. DOSS, Lexington, KY (US); Kevin Paul ROSENBLATT, Bellaire, TX (US)

(21) Appl. No.: 16/945,644

(22) Filed: Jul. 31, 2020

Related U.S. Application Data
(63) Continuation of application No. PCT/US2019/016192, filed on Jan. 31, 2019.
(60) Provisional application No. 62/624,713, filed on Jan. 31, 2018.
(60) Provisional application No. 62/796,557, filed on Jan. 24, 2019.

(51) Int. Cl. G16B 5/20; G16B 40/00; G16H 50/30; G06N 20/00

(52) U.S. Cl. CPC G16B 5/20; G06N 20/00; G16H 50/30; G16B 40/00

(57) ABSTRACT

The present disclosure relates to systems and methods of using machine learning analysis to stratify the risk of spontaneous preterm birth (SPTB). In some variations, to select informative markers that differentiate SPTB from term deliveries, a processed quantification data of the markers can be subjected to univariate receiver operating characteristic (ROC) curve analysis. A Differential Dependency Network (DDN) can then applied in order to extract co-expression patterns among the markers. In order to assess the complementary values among selected markers and the range of their relevant performance, multivariate linear models can be derived and evaluated using bootstrap resampling.

### CROSS-REFERENCE TO RELATED APPLICATIONS

This application claims the benefit under 35 U.S.C. § 119(e) of U.S. Provisional Patent Application No. 62/624,713, filed Jan. 31, 2018, and U.S. Provisional Patent Application No. 62/796,557, filed Jan. 24, 2019. The contents of these applications are incorporated herein by reference in their entireties.

### BACKGROUND

Preterm birth is a leading cause of neonatal morbidity and death in children less than 5 years of age, with deliveries at the earlier gestational ages exhibiting a dramatically increased risk (Liu et al., Lancer, 385:61698-61706, 2015; and Katz et al., Lancet, 382:417-425, 2013). Compared with infants born after 38 weeks, the composite rate of neonatal morbidity doubles for each earlier gestational week of delivery according to the March of Dimes. Approximately two thirds of spontaneous preterm births (SPTBs) are spontaneous in nature, meaning they are not associated with medical intervention (Goldenberg et al., Lancet, 371:75-84, 2008; and McElrath et al., Am J Epidemiol, 168:980-989, 2008). Yet, despite the compelling nature of this condition, there has been little recent advancement understanding of the etiology of spontaneous preterm birth (SPTB). While there is an increasing consensus that SPTB represents a syndrome rather than a single pathologic entity, it has been both ethically and physically difficult to study the pathophysiology of the utero-placental interface (Romero et al., Science, 345:760-765, 2014). The evolving field of circulating microparticle (CMP) biology may offer a solution to these difficulties as these particles present a sampling of the utero-placental environment. Additionally, studying the contents of these particles holds the promise of identifying novel blood-based, and clinically useful, biomarkers.

Microparticles are membrane-bound vesicles that range in size from 50-300 nm and shed by a wide variety of cell types. Microparticle nomenclature varies, but typically microparticles between 50-100 nm are called exosomes, those >100 nm are termed microvesicles and other terms, such as microaggregates, are often used in literature. Unless otherwise stated, the term microparticle is a general reference to all of these species. Increasingly, microparticles are recognized as important means of intercellular communication in physiologic, pathophysiologic and apoptotic circumstances. While the contents of different types of microparticles vary with cell type, they can include nuclear, cytosolic and membrane proteins, as well as lipids and messenger and micro RNAs. Information regarding the state of the cell type of origin can be derived from an examination of microparticle contents. Thus, microparticles represent an unique window in real-time into the activities of cells, tissues and organs that may otherwise be difficult to sample.

A high proportion of adverse pregnancy outcomes have their pathophysiologic origins at the utero-placental interface in early pregnancy (Romero et al., supra, 2014; Gagnon, Eur J Obstet Gynecol Reprod Biol, 110:S99-S107, 2003; and Masoura et al., J. Obstet Gynaecol, 32:609-616, 2012). The ability to assess the state of associated tissue and cell populations is expected to be predictive of impending complications. Noninvasive tools for discriminating between pregnancies delivering at gestational ages marked by considerable neonatal morbidity (<34 weeks or <35) compared with those delivering at term are particularly desirable given that timely administration of therapeutic agents may prevent preterm labor or otherwise prolong pregnancy.

Much needed are tools for determining whether a pregnant woman is at an increased risk for premature delivery, as well as tools for decreasing a pregnant subject's risk for premature delivery. Provided herein are such tools.

Patents, patent applications, patent application publications, journal articles and protocols referenced herein are incorporated by reference.

### BRIEF SUMMARY OF THE INVENTION

The present disclosure relates to proteomic biomarkers of SPTB, proteomic biomarkers of term birth, and methods of use thereof. In particular, the present disclosure provides tools for determining whether a pregnant subject is at an increased risk for premature delivery, as well as tools for decreasing a pregnant subject's risk for premature delivery.

In one aspect provided herein is a method for assessing risk of spontaneous preterm birth for a pregnant subject, the method comprising:

(a) preparing a microparticle-enriched fraction from a blood sample from the pregnant subject; and
(b) determining a quantitative measure of a panel of microparticle-associated proteins in the fraction, wherein the panel comprises ICI, ITIH4, and LCAT.
In some embodiments, the panel further comprises a fourth protein. In some embodiments, the fourth protein is TRFE. In some embodiments, the panel comprises the proteins IC1, ITIH4, LCAT, and TRFE. In some embodiments, the panel consists of the proteins IC1, ITIH4, LCAT, and TRFE. In some embodiments, the pregnant subject is primiparous. In some embodiments, the blood sample is taken from the pregnant subject when the pregnant human subject is at 10 to 12 weeks of gestation. In some embodiments, the blood sample is taken from the pregnant subject during the first trimester of gestation. In some embodiments, the method assesses the risk of the pregnant subject having a greater likelihood of having a spontaneous preterm birth at or before 35 weeks of gestation.

In another aspect, provided herein is a method for assessing risk of spontaneous preterm birth for a pregnant subject, the method comprising:

(a) preparing a microparticle-enriched fraction from a blood sample from the pregnant subject; and
(b) determining a quantitative measure of a panel of microparticle-associated proteins in the fraction, wherein the panel comprises F13A, FBLN1, ICI, LCAT and one protein selected from ITIH1 or ITIH2.
In some embodiments, the panel comprises F13A, FBLN1, ICI, LCAT and ITIH1. In some embodiments, the panel panel comprises F13A, FBLN1, ICI, LCAT and ITIH2. In some embodiments, the panel panel consists of F13A, FBLN1, ICI, LCAT and ITIH1. In some embodiments, the panel panel consists of F13A, FBLN1, ICI, LCAT and ITIH2. In some embodiments, the panel pregnant subject is multiparous. In some embodiments, the panel pregnant subject is primiparous. The In some embodiments, the panel pregnant subject is primigravida. In some embodiments, the panel pregnant subject is multigravida. In some embodiments, the panel blood sample is taken from the pregnant subject when the pregnant human subject is at 10 to 12 weeks of gestation. In some embodiments, the panel blood sample is taken from the pregnant subject during the first trimester of gestation. In some embodiments, the panel method assesses the risk of the pregnant subject having a greater likelihood of having a spontaneous preterm birth at or before 35 weeks of gestation.

In another aspect, provided herein is a method for assessing the likelihood of a pregnant subject having a spontaneous preterm birth at or before 35 weeks of gestation, the method comprising:

(a) preparing a microparticle-enriched fraction from a blood sample from the pregnant subject; and
(b) determining a quantitative measure of a panel of microparticle-associated proteins in the fraction, wherein the panel either (i) comprises IC1, ITIH4, LCAT, and TRFE, or (ii) consists of IC1, ITIH4, LCAT, and TRFE, wherein the pregnant subject is primiparous, and wherein the blood sample is taken from the pregnant subject when the pregnant human subject is at 10 to 12 weeks of gestation.

In a related aspect, provided herein is a method for assessing the likelihood of a pregnant subject having a spontaneous preterm birth at or before 35 weeks of gestation, the method comprising:

(a) preparing a microparticle-enriched fraction from a blood sample from the pregnant subject; and
(b) determining a quantitative measure of a panel of microparticle-associated proteins in the fraction, wherein the panel either (i) comprises F13A, FBLN1, ICI, LCAT and ITIH2, or (ii) consists of F13A, FBLN1, ICI, LCAT and ITIH2, wherein the pregnant subject is primiparous, and wherein the blood sample is taken from the pregnant subject when the pregnant human subject is at 10 to 12 weeks of gestation.
In some embodiments of either of the above two aspects, the steps of the method are carried out on a first sample taken from the pregnant subject during the first trimester, and the steps of the method are repeated on a second sample taken from the pregnant subject during the second trimester. In some embodiments, the steps of the method are carried out on a first sample taken from the pregnant subject at 8 to 12 weeks of gestation, and the steps of the method are repeated on a second sample taken from the pregnant subject at 18 to 24 weeks of gestation. In some embodiments, the steps of the method are carried out on a first sample taken from the pregnant subject at 10 to 12 weeks of gestation, the steps of the method are repeated on a second sample taken from the pregnant subject during the second trimester. In some embodiments, the steps of the method are carried out on a first sample taken from the pregnant subject at 10 to 12 weeks of gestation, the steps of the method are repeated on a second sample taken from the pregnant subject at 18 to 24 weeks of gestation. In some embodiments, the blood sample is a serum sample. In some embodiments, the blood sample is a plasma sample. In some embodiments, the microparticle-enriched fraction is prepared using size-exclusion chromatography. In some embodiments, the size-exclusion chromatography comprises elution with water. In some embodiments, the size-exclusion chromatography is performed with an agarose solid phase and an aqueous liquid phase. In some embodiments, the preparing step further comprises using ultrafiltration or reverse-phase chromatography. In some embodiments, the preparing step further comprises denaturation using urea, reduction using dithiothreitol, alkylation using iodoacetamine, and digestion using trypsin prior to the size exclusion chromatography. In some embodiments, the determining a quantitative measure of a panel of microparticle-associated proteins in the fraction comprises detection of any one or more of the peptides presented in Table 14A or comprises detection of any one or more of the peptides presented in Table 14B. In some embodiments, the determining a quantitative measure of a panel of microparticle-associated proteins in the fraction comprises detecting peptides represented by SEQ ID NO:1, SEQ ID NO:2, SEQ ID NO:3, and SEQ ID NO:4, wherein the pregnant subject is primiparous, and wherein the blood sample is taken from the pregnant subject when the pregnant human subject is at 10 to 12 weeks of gestation. In some embodiments, the determining a quantitative measure of a panel of microparticle-associated proteins in the fraction comprises detecting peptides represented by SEQ ID NO:5, SEQ ID NO:6, SEQ ID NO:1, SEQ ID NO:7, and SEQ ID NO:2, wherein the pregnant subject is primiparous or multiparous, and wherein the blood sample is taken from the pregnant subject when the pregnant human subject is at 10 to 12 weeks of gestation. In some embodiments, the determining a quantitative measure of a panel of microparticle-associated proteins in the fraction comprises mass spectrometry. In some embodiments, the determining a quantitative measure of a panel of microparticle-associated proteins in the fraction comprises liquid chromatography/mass spectrometry. In some embodiments, the mass spectrometry comprises multiple reaction monitoring, the liquid chromatography is performed using a solvent comprising acetonitrile, and/or the detecting step comprises assigning an indexed retention time to the proteins. In some embodiments, the determining a quantitative measure of a panel of microparticle-associated proteins in the fraction comprises mass spectrometry/multiple reaction monitoring (MS/MRM). In some embodiments, the MS/MRM involves the use of a plurality of stable isotope standards. In some embodiments, the MS/MRM involves the use of a plurality of stable isotope standards provided in Table 15A or Table 15B. In some embodiments, the determining comprises executing a classification rule, which rule classifies the subject at being at risk of spontaneous preterm birth, and wherein execution of the classification rule produces a correlation between preterm birth or term birth with a p value of less than at least 0.05. In some embodiments, the determining comprises executing a classification rule, which rule classifies the subject at being at risk of spontaneous preterm birth, and wherein execution of the classification rule produces a receiver operating characteristic (ROC) curve, wherein the ROC curve has an area under the curve (AUC) of at least 0.6. In some embodiments, the values on which the classification rule classifies a subject further include at least one of: maternal age, maternal body mass index, parity status, and smoking during pregnancy. The In some embodiments, the classification rule is configured to have a specificity of at least 80%, at least 90% or at least 95%. In some embodiments, the method further comprises a treatment step selected from the group consisting of a hormone and a corticosteroid.

In another aspect, provided herein is a method of decreasing risk of spontaneous preterm birth for a pregnant subject and/or reducing neonatal complications of spontaneous preterm birth, the method comprising:

(a) assessing risk of spontaneous preterm birth for a pregnant subject according to any of the method provided herein; and
(b) administering a therapeutic agent to the subject in an amount effective to decrease the risk of spontaneous preterm birth and/or reduce neonatal complications of spontaneous preterm birth.
In some embodiments, the therapeutic agent is selected from the group consisting of a hormone and a corticosteroid. In some embodiments, the therapeutic agent comprises vaginal progesterone or parenteral 17-alpha-hydroxyprogesterone caproate.

In another aspect, provided herein is a method comprising administering to a pregnant subject characterized as having a panel of microparticle-associated proteins indicative of an increased risk of spontaneous preterm birth, an effective amount of a treatment designed to reduce the risk of spontaneous preterm birth, wherein the panel comprises IC1, ITIH4, LCAT, and TRFE or the panel comprises F13A, FBLN1, ICI, LCAT and ITIH2.

In another aspect, provided herein is a method comprising administering to a pregnant subject characterized as having a panel of microparticle-associated proteins indicative of an increased risk of spontaneous preterm birth, an effective amount of a treatment designed to reduce the risk of spontaneous preterm birth, wherein the panel consists of IC1, ITIH4, LCAT, and TRFE or the panel consists of F13A, FBLN1, ICI, LCAT and ITIH2. In some embodiments, the treatment is selected from the group consisting of a hormone and a corticosteroid. In some embodiments, the treatment comprises vaginal progesterone or parenteral 17-alpha-hydroxyprogesterone caproate. In some embodiments, the pregnant subject is primiparous. In some embodiments, the blood sample is taken from the pregnant subject when the pregnant human subject is at 10 to 12 weeks of gestation.

In another aspect provided herein is a method of decreasing risk of spontaneous preterm birth for a pregnant subject and/or reducing neonatal complications of spontaneous preterm birth, the method comprising:

(a) assessing risk of spontaneous preterm birth for a pregnant subject according to the any of the method provided herein; and
(b) administering a therapeutic agent to the subject in an amount effective to decrease the risk of spontaneous preterm birth and/or reduce neonatal complications of spontaneous preterm birth.

In another aspect, provided herein is a method comprising:

(a) preparing a microparticle-enriched fraction from plasma or serum of a pregnant subject at from 8 to 14 weeks of gestation;
(b) using selected reaction monitoring mass spectrometry, determining a quantitative measure of a panel of proteins in the fraction, wherein the panel (i) comprises IC1, ITIH4, LCAT, and TRFE; (ii) comprises F13A, FBLN1, ICI, LCAT and ITIH2; (iii) consists of IC1, ITIH4, LCAT, and TRFE; or (iv) consists of F13A, FBLN1, ICI, LCAT and ITIH2; and
(c) executing a classification rule of a classification system which rule, based on values including the quantitative measures, classifies the subject as being at risk of spontaneous preterm birth, wherein the classification system, in a receiver operating characteristic (ROC) curve, has an area under the curve (AUC) of at least 0.6.

In another aspect, provided herein is a method of decreasing risk of spontaneous preterm birth and/or reducing neonatal complications, the method comprising:

(a) determining by any of the of methods provided herein that a subject is at risk of spontaneous preterm birth; and
(b) administering to the subject a therapeutic agent in an amount effective to decrease the risk of spontaneous preterm birth and/or reduce neonatal complications.

In another aspect, provided herein is a method comprising:

(a) providing a microparticle-enriched fraction from plasma or serum of a plurality of pregnant subjects obtained at from 8 to 14 weeks of gestation, wherein the plurality of subjects include a plurality of subjects that subsequently experienced preterm birth and a plurality of subjects that subsequently experienced term birth;
(b) using selected reaction monitoring mass spectrometry, determining a quantitative measure of a panel of proteins in the fraction, wherein the panel (i) comprises IC1, ITIH4, LCAT, and TRFE; (ii) comprises F13A, FBLN1, ICI, LCAT and ITIH2; (iii) consists of IC1, ITIH4, LCAT, and TRFE; or (iv) consists of F13A, FBLN1, ICI, LCAT and ITIH2;
(c) preparing a training data set indicating, for each sample, values indicating: (i) classification of the sample as belonging to preterm birth or term birth classes; and (ii) the quantitative measures of the plurality of protein biomarkers; and
(d) training a learning machine algorithm on the training data set, wherein training generates one or more classification rules that classify a sample as belonging to the preterm birth class or the term birth class.

In another aspect, provided herein is a method for measuring a protein panel, comprising:

(a) preparing a sample comprising proteins from a microparticle-enriched fraction of a blood sample; performing protease digestion on the proteins to produce peptide fragments; contacting the peptide fragments with a plurality of isotope-labeled reference peptides comprising, or consisting of SEQ ID NO:8 SEQ ID NO:9, SEQ ID NO:10, and SEQ ID NO:11;
(b) determining a quantitative measure of a panel of microparticle-associated proteins in the fraction, wherein the panel comprises or consists of ICI, ITIH4, TRFE, and LCAT. In some embodiments, the method comprises using MS/MRM to perform the method. In some embodiments, the blood sample comprises a plasma sample. In some embodiments, the blood sample comprises a serum sample. In some embodiments, the blood sample is from a subject, and the subject is a pregnant subject who is at 8 to 14 weeks of gestation. In some embodiments, the blood sample is from a subject, and the subject is a pregnant subject who is at 10 to 12 weeks of gestation. In some embodiments, the blood sample is from a subject, and the subject is a pregnant subject who is primiparous.

In another aspect provided herein is a method for measuring a protein panel, comprising:

(a) preparing a sample comprising proteins from a microparticle-enriched fraction of a blood sample;
(b) performing protease digestion on the proteins to produce peptide fragments;
(c) contacting the peptide fragments with a plurality of isotope-labeled reference peptides comprising, or consisting of SEQ ID NO:12, SEQ ID NO:13, SEQ ID NO:8, SEQ ID NO:14, and SEQ ID NO:9; and
(d) determining a quantitative measure of a panel of microparticle-associated proteins in the fraction, wherein the panel comprises or consists of F13A, FBLN1, ICI, ITIH1, and LCAT. In some embodiments, the method comprises using MS/MRM to perform the method. In some embodiments, the blood sample comprises a plasma sample. In some embodiments, the blood sample comprises a serum sample. In some embodiments, the blood sample is from a subject, and the subject is a pregnant subject who is at 8 to 14 weeks of gestation. In some embodiments, the blood sample is from a subject, and the subject is a pregnant subject who is at 10 to 12 weeks of gestation. In some embodiments, the blood sample is from a subject, and the subject is a pregnant subject who is primiparous.

In another aspect, provided herein is a method for measuring a protein panel, comprising:

(a) preparing a microparticle-enriched fraction from a blood sample of a subject; and
(b) determining a quantitative measure of a panel of microparticle-associated proteins in the fraction, wherein the panel comprises or consists of F13A, FBLN1, ICI, ITIH1, and LCAT, and wherein the determining comprises measuring surrogate peptides of the proteins.
In some embodiments, the method comprises measuring the level of the surrogate peptide sequences of SEQ ID NO:5, SEQ ID NO:6, SEQ ID NO:1, SEQ ID NO:7, and SEQ ID NO:2. In some embodiments, the method comprises using MS/MRM to perform the method. In some embodiments, the method further comprises using the isotope-labeled reference peptides of SEQ ID NO:12, SEQ ID NO:13, SEQ ID NO:8, SEQ ID NO:14, and SEQ ID NO:9. In some embodiments, the blood sample comprises a plasma sample. In some embodiments, the blood sample comprises a serum sample. In some embodiments, the subject is a pregnant subject who is at 8 to 14 weeks of gestation. In some embodiments, the subject is a pregnant subject who is at 10 to 12 weeks of gestation. In some embodiments, the subject is a pregnant subject who is primiparous. In some embodiments, the subject is a pregnant subject who is multiparous.

In another aspect, provided herein is a method for measuring a protein panel, comprising:

(a) preparing a microparticle-enriched fraction from a blood sample of a subject; and
(b) determining a quantitative measure of a panel of microparticle-associated proteins in the fraction, wherein the panel comprises or consists of ICI, ITIH4, TRFE, and LCAT, and wherein the determining comprises measuring surrogate peptides of the proteins. In some embodiments, the method comprises measuring the level of the surrogate peptide sequences of SEQ ID NO:1, SEQ ID NO:2, SEQ ID NO:3, and SEQ ID NO:4.
In some embodiments, the method comprises using MS/MRM to perform the method. In some embodiments, the method comprises using the isotope-labeled reference peptides of SEQ ID NO:8 SEQ ID NO:9, SEQ ID NO:10, and SEQ ID NO:11. In some embodiments, the blood sample comprises a plasma sample. In some embodiments, the blood sample comprises a serum sample. In some embodiments, the subject is a pregnant subject who is at 8 to 14 weeks of gestation. In some embodiments, the subject is a pregnant subject who is at 10 to 12 weeks of gestation. In some embodiments, the subject is a pregnant subject who is primiparous.

In another aspect, provided herein is a method for measuring a protein panel, comprising:

(a) preparing a microparticle-enriched fraction from a blood sample of a subject; and
(b) determining a quantitative measure of a panel of microparticle-associated proteins in the fraction, wherein the panel comprises or consists of F13A, FBLN1, ICI, ITIH1, and LCAT, and wherein the determining comprises measuring surrogate peptides of the proteins.
In some embodiments, the method comprises measuring the level of the surrogate peptide sequences of SEQ ID NO:5, SEQ ID NO:6, SEQ ID NO:1, SEQ ID NO:7, and SEQ ID NO:2. In some embodiments, the method comprises using MS/MRM to perform the method. In some embodiments, the method comprises further comprises using the isotope-labeled reference peptides of SEQ ID NO:12, SEQ ID NO:13, SEQ ID NO:8, SEQ ID NO:14, and SEQ ID NO:9. In some embodiments, the blood sample comprises a plasma sample. In some embodiments, the blood sample comprises a serum sample. In some embodiments, the subject is a pregnant subject who is at 8 to 14 weeks of gestation. In some embodiments, the subject is a pregnant subject who is at 10 to 12 weeks of gestation. In some embodiments, the subject is a pregnant subject who is primiparous. In some embodiments, the subject is a pregnant subject who is multiparous.

In another aspect, provided herein is a kit comprising for measuring spontaneous preterm birth in a pregnant subject comprising the isotope-labeled reference peptides of SEQ ID NO:8, SEQ ID NO:9, SEQ ID NO:10, and SEQ ID NO:11, and instructions for use.

In another aspect, provided herein is a kit comprising for measuring spontaneous preterm birth in a pregnant subject comprising the isotope-labeled reference peptides of SEQ ID NO:12, SEQ ID NO:13, SEQ ID NO:8, SEQ ID NO:14, and SEQ ID NO:9, and instructions for use.

In another aspect, provided herein is a composition comprising a plurality of protein peptides and a plurality of isotope-labeled reference peptides, wherein the protein peptides comprise, or consist of SEQ ID NO:1, SEQ ID NO:2, SEQ ID NO:3, and SEQ ID NO:4 and the isotope-labeled reference peptides comprise or consist of SEQ ID NO:8 SEQ ID NO:9, SEQ ID NO:10, and SEQ ID NO:11

In another aspect, provided herein is a composition comprising a plurality of protein peptides and a plurality of isotope-labeled reference peptides, wherein the protein peptides comprise, or consist of SEQ ID NO:5, SEQ ID NO:6, SEQ ID NO:1, and SEQ ID NO:7, and SEQ ID NO:2 and the isotope-labeled reference peptides comprise or consist of SEQ ID NO:12, SEQ ID NO:13, SEQ ID NO:8, SEQ ID NO:14, and SEQ ID NO:9.

In another aspect, provided herein is a computer system comprising: a processor; and a memory, coupled to the processor, the memory storing a module comprising:

(i) test data for a sample from a subject including values indicating a quantitative measure of a panel of protein biomarkers in the fraction, wherein the panel (i) comprises IC1, ITIH4, LCAT, and TRFE; (ii) comprises F13A, FBLN1, ICI, LCAT and ITIH2; (iii) consists of IC1, ITIH4, LCAT, and TRFE; or (iv) consists of F13A, FBLN1, ICI, LCAT and ITIH2;
(ii) a classification rule which, based on values including the measurements, classifies the subject as being at risk of pre-term birth, wherein the classification rule is configured to have a sensitivity of at least 75%, at least 85% or at least 95%; and
(iii) computer executable instructions for implementing the classification rule on the test data.

### BRIEF DESCRIPTION OF THE DRAWINGS

FIG. 1 is a graph of a bootstrap ROC analysis to select proteins for detection of SPTBs from term cases. Each protein was plotted as a blue-colored point with mean and SD of the AUCs from bootstrap ROC analysis as x- and y-axis values, correspondingly. Results from the same analysis yet with sample label permutation were plotted as red points. A total of 62 proteins (blue points) within the lower right quadrant bounded by the magenta vertical line (mean+SD of x-values of the red points) and the green horizontal line (mean+SD of y values of the blue points) were selected for their relatively stable and significant discriminatory power. In comparison, only 12 of proteins from label permutated analysis (red points) were in this quadrant. The estimated false discovery rate was therefore <20% ( 12/62).

FIG. 2 illustrates a Differential Dependency Network (DDN) analysis of selected proteins identified as having co-expression patterns associated with STPB. In the plot, red lines indicate that co-expression between the pairs of proteins were observed among STPBs, while green lines indicate that co-expression between the pairs of proteins were observed among the TERM cases. The thickness of the lines is proportional to the statistical significance of the connection.

FIG. 3 shows the frequency of DDN-selected proteins in top 20 multivariate models based on AUC in Table 7 (top) or specificity at a fixed sensitivity of 80% in Table 8 (bottom).

FIG. 4A and FIG. 4B show ROC curves of exemplary linear models combining three proteins. ROC analysis with bootstrap resampling provided an estimated range of performance in training data.

FIG. 4C shows the frequency of marker inclusion in the top 100 panels of five to eight microparticle-associated proteins.

FIG. 5 shows temporal patterns in protein expression over two time points (D1=about 10-12 weeks gestation; D2=about 22-24 weeks gestation) carries differential information between SPTBs and controls.

FIG. 6 shows a selection of proteins for SPTB detection.

FIG. 7 shows proteins with statistically consistent performance.

FIG. 8 shows that 2 pools in SEC data from samples in Example 2 demonstrate high analytical precision (small coefficient of variation).

FIG. 9 shows the of NeXosome® sample prep step (SEC) on number of proteins informative in detecting SPTB from controls, from samples used in Example 2.

FIG. 10 shows the effect of SEC on concentration of abundant protein ALBU.

FIG. 11 shows that SEC improved separation between SPTB and controls in discrimination the biomarker ITIH4 in samples taken at 22-24 weeks gestation.

FIG. 12A and FIG. 12B show the performance of one exemplary 5 protein marker panel, optimized for all subjects regardless of parity status or other factors such as fetal gender.

FIG. 12C shows the performance of another exemplary 5 protein marker panel, also optimized for all subjects regardless of parity status or other factors such as fetal gender.

FIG. 12D shows that test performance varied based on fetal sex and parity.

FIG. 13 shows the consistency and stability of markers over multiple iterations, supporting the selection of the exemplary 5 protein marker panels, for example those shown in FIGS. 12A, 12B, and 12C.

FIG. 14 shows the performance of a multivariate model optimized for parity=0 with a 4 protein marker panel.

FIG. 15 shows the performance of a 4 protein marker panel by fetal gender.

FIG. 16 shows Kaplan-Meier curves for pregnancy survival by week of gestation using the multi-marker panel selected for the primipara (parity=0) mothers in FIG. 15, and classifying the pregnancies into high and low risk strata across the test set.

FIG. 17 shows 5-marker panels and their training/cross-validation performance of some of the top performing panels in terms of mean and standard deviation of AUC, with the sensitivity at a prefixed specificity (0.65) and specificity at prefixed sensitivity (0.75).

### DETAILED DESCRIPTION OF THE INVENTION

This disclosure provides statistically significant CMP-associated (circulation microparticle-associated) protein biomarkers and multiplex panels associated with biological processes relevant to pregnancy that are already unique in their expression profiles at 10-12 weeks gestation among females who go on to deliver spontaneously at <38 weeks (e.g. at <35 weeks). These biomarkers are useful for the clinical stratification of patients at risk of SPTB well before clinical presentation. Such identification is indicative of a need for increased observation and may result in the application of prophylactic therapies, which together may significantly improve the management of these patients.

### Protein Biomarker Panels

The present disclosure provides tools for assessing and decreasing risk of SPTB. The methods of the present disclosure include a step of detecting the level of at least one microparticle-associated protein in a biological sample.

A microparticle refers to an extracellular microvesicle or lipid raft protein aggregate having a hydrodynamic diameter of from about 50 to about 5000 nm. As such the term microparticle encompasses exosomes (about 50 to about 100 nm), microvesicles (about 100 to about 300 nm), ectosomes (about 50 to about 1000 nm), apoptotic bodies (about 50 to about 5000 nm) and lipid protein aggregates of the same dimensions. As used herein, the term “about” as used herein in reference to a value refers to 90 to 110% of that value. For instance a diameter of about 1000 nm is a diameter within the range of 900 nm to 1100 nm.

A microparticle-associated protein refers to a protein or fragment thereof (e.g., polypeptide) that is detectable in a microparticle-enriched sample from a mammalian (e.g., human) subject. As such a microparticle-associated protein is not restricted to proteins or fragments thereof that are physically associated with microparticles at the time of detection; the proteins or fragments may be incorporated between microparticles, or the proteins or fragments may have been associate with the microparticle at some earlier time prior to detection.

Unless otherwise stated, the term protein encompasses polypeptides and fragments thereof. “Fragments” include polypeptides that are shorter in length than the full length or mature protein of interest. If the length of a protein is x amino acids, a fragment is x−1 amino acids of that protein. The fragment may be shorter than this (e.g., x−2, x−3, x−4, . . . ), and is preferably 100 amino acids or less (e.g., 90, 80, 70, 60, 50, 40, 30, 20 or 10 amino acids or less). The fragment may be as short as 4 amino acids, but is preferably longer (e.g., 5, 6, 7, 8, 9, 10, 12, 15, 20, 25, 30, 35, 40, 50, 60, 70, 80, 90, or 100 amino acids). In exemplary embodiments, a plurality of surrogate peptides indicative of the presence of a set of biomarkers are quantified.

The present disclosure provides tools for detecting the level of at least one microparticle-associated protein, more preferably at least three, four or five proteins. The disclosure however is focused on exemplary combination of a four-protein panel that is highly predictive of SPTB in a nulliparous pregnant subject and another exemplary combination of a five-protein panel that is highly predictive of SPTB irrespective of parity of the pregnant subject

As used herein “detecting the level” of at least one microparticle-associated protein encompasses detecting the expression level of the protein, detecting the absolute concentration of the protein, detecting an increase or decrease of the protein level in relation to a reference standard, detecting an increase or decrease of the protein level in relation to a threshold level, measuring the protein concentration, quantifying the protein concentration, determining a quantitative measure, detecting the presence (e.g., level above a threshold or detectable level) or detecting the absence (e.g., level below a threshold or undetectable level) of at least one microparticle-associated protein in a sample from a pregnant subject. In some embodiments, the quantitative measure can be an absolute value, a ratio, an average, a median, or a range of numbers.

As used herein, “detection of a protein” and “determining a quantitative measure of one or more proteins” encompasses any means, including, detection by an MS method that detects fragments of a protein. The data disclosed in the tables and figures was obtained by MRM-MS, which detects proteins by selecting peptide fragments of a parent protein for detection as surrogates—exemplary surrogate peptides of the disclosure are provided in Tables 14A and 14B.

During development of the present disclosure numerous microparticle-associated proteins were determined to be altered in samples from subjects having preterm births (as compared to samples from subjects have term births), and are therefore termed “preterm birth biomarkers.” Additionally during development of the present disclosure numerous microparticle-associated proteins were determined to be not altered in samples from subjects having preterm births (as compared to samples from subjects have term births), and are therefore termed “term birth biomarkers.” More specifically, a discrete four biomarker was surprisingly found to be predictive of SPTB in nulliparous pregnant subjects (ICI, ITIH4, TRFE, and LCAT). Equally surprisingly a discrete five biomarker panel was found to be predictive of SPTB in pregnant subjects regardless of parity (F13A, FBLN1, ICI, ITIH1, and LCAT).

Accordingly, in some exemplary embodiments, the methods of the present disclosure include a step of detecting the level of a panel of microparticle-associated proteins in a biological sample from a nulliparous pregnant test subject who is at 8-14 weeks, or at 10-12 weeks of gestation, where the microparticle-associated proteins comprise ICI, ITIH4, TRFE, and LCAT. In some exemplary embodiments, the methods of the present disclosure include a step of detecting the level of a panel of microparticle-associated proteins in a biological sample from a nulliparous pregnant test subject, where the microparticle-associated proteins consist of ICI, ITIH4, TRFE, and LCAT.

Accordingly, in some exemplary embodiments, the methods of the present disclosure include a step of detecting the level of a panel of microparticle-associated proteins in a biological sample from a nulliparous or multiparous pregnant test subject who is at 8-14 weeks, or at 10-12 weeks of gestation, where the microparticle-associated proteins comprise F13A, FBLN1, ICI, ITIH1, and LCAT. In some exemplary embodiments, the methods of the present disclosure include a step of detecting the level of a panel of microparticle-associated proteins in a biological sample from a nulliparous or multiparous pregnant test subject, where the microparticle-associated proteins consist of F13A, FBLN1, ICI, ITIH1, and LCAT.

In other embodiments, the methods of the present disclosure include a step of detecting the level of a panel of microparticle-associated proteins in a biological sample from a pregnant test subject, where the microparticle-associated proteins are from Table 1. In some embodiments, the methods of the present disclosure include a step of detecting the level of at least one microparticle-associated protein in a biological sample from a pregnant test subject, where the at least one protein is selected from Table 1. In some embodiments, the methods of the present disclosure include a step of detecting the level of at least two, at least three, at least four, at least five, at least six, at least seven, at least eight, at least nine, or at least ten microparticle-associated proteins in a biological sample from a pregnant test subject, where the at least one protein is selected from Table 1. In some embodiments, the methods of the present disclosure include a step of detecting the level of five, six, seven, eight, or nine microparticle-associated proteins in a biological sample from a pregnant test subject, where the proteins are selected from Table 1. In an exemplary embodiment, the methods of the present disclosure include a step of detecting the level of six microparticle-associated proteins in a biological sample from a pregnant test subject, where the six proteins are selected from Table 1. In an exemplary embodiment, the methods of the present disclosure include a step of detecting the level of seven microparticle-associated proteins in a biological sample from a pregnant test subject, where the seven proteins are selected from Table 1. In an exemplary embodiment, the methods of the present disclosure include a step of detecting the level of eight microparticle-associated proteins in a biological sample from a pregnant test subject, where the eight proteins are selected from Table 1. In an exemplary embodiment, the methods of the present disclosure include a step of detecting the level of nine microparticle-associated proteins in a biological sample from a pregnant test subject, where the nine proteins are selected from Table 1.

In some embodiments, if the sample is obtained at about 10-12 weeks gestation, the microparticle-associated protein can display the directionality (+ or −) indicated in the last column of Table 1. In the last column of Table 1, (−) indicates the biomarker is downregulated in SPTB cases versus TERM controls; and (+) indicates the biomarker is upregulated in SPTB cases vs TERM controls.

TABLE 1
Microparticle-Associated Proteins Differentially
Expressed in Preterm Birth

| | | | (+ |
|---|---|---|---|
| | | | or −) |
| | | | 10-12 |
| Symbol | Protein Name (Alternative Name) | UniProtKB | wk |
| A1AG1 | Alpha-1-acid glycoprotein 1 | P02763 | − |
| (ORM1) | (Orosomucoid-1) | | |
| A1AG2 | Alpha-1-acid glycoprotein 2 | P19652 | − |
| (ORM2) | (Orosomucoid-2) | | |
| A1AT | Alpha-1-antitrypsin | P01009 | − |
| (SERPINA1) | | | |
| A1BG | Alpha-1B-glycoprotein | P04217 | − |
| A2AP | Alpha-2-antiplasmin | P08697 | |
| (SERPINF2) | | | |
| A2GL (LRG) | Leucine-rich alpha-2-glycoprotein | P02750 | − |
| A2MG (A2M) | Alpha-2-macroglobulin | P01023 | − |
| AACT | Alpha-1-antichymotrypsin | P01011 | − |
| (SERPINA3) | | | |
| AMBP | Alpha-1-microglobulin/bikunin | P02760 | − |
| | precursor | | |
| ANGT | Angiotensinogen | P01019 | + |
| (SERPINA8) | | | |
| ANT3 | Antithrombin-III | P01008 | − |
| (SERPINC1) | | | |
| APOA1 | Apolipoprotein A1 | P02647 | − |
| APOA4 | Apolipoprotein A1 | P06727 | − |
| APOB | Apolipoprotein B100 | P04114 | + |
| APOC3 | Apolipoprotein C3 | P02656 | + |
| APOD | Apolipoprotein D | P05090 | − |
| APOE | Apolipoprotein E | P02649 | + |
| APOH | Apolipoprotein H | P02749 | + |
| APOL1 | Apolipoprotein L1 | O14791 | − |
| APOM | Apolipoprotein M | O95445 | − |
| ATRN | Attractin | O75882 | − |
| BTD | Biotinidase | P43251 | − |
| C1QA | Complement C1q subunit A | P02745 | + |
| C1QC | Complement C1q subunit C | P02747 | − |
| C1R | Complement C1r | P00736 | − |
| C1S | Complement C1s | P09871 | + |
| C4BPA | Complement C4b-binding protein | P04003 | + |
| | alpha chain | | |
| C6 | Complement C6 (CO6) | P13671 | − |
| C8A | Complement C8 alpha chain | P07357 | − |
| | (CO8A) | | |
| C8G | Complement C8 gamma chain | P07360 | − |
| | (CO8G) | | |
| C9 | Complement C9 (CO9) | P02748 | − |
| CBG | Corticosteroid-binding globulin | P08185 | + |
| (SERPINA6) | | | |
| CD5L | CD5 antigen-like | O43866 | − |
| CERU (CP) | Ceruloplasmin (Ferroxidase) | P00450 | + |
| CFAB (CFB) | Complement Factor B (C3/C5 | P00751 | − |
| | convertase) | | |
| CFAD (CFD) | Complement Factor D (Adipsin) | P00746 | + |
| CFAI (CFI) | Complement Factor I (C3B/C4B | P05156 | + |
| | inact.) | | |
| CHLE | Cholinesterase | P06276 | − |
| CLUS | Clusterin (Apolipoprotein J) | P10909 | − |
| CPN1 (CBPN) | Carboxypeptidase N, polypeptide 1 | P15169 | − |
| CPN2 | Carboxypeptidase N, polypeptide 2 | P22792 | − |
| F10 (FA10) | Coagulation factor X | P00742 | − |
| F12 (FA12) | Coagulation factor XII | P00748 | − |
| F13A | Coagulation factor XIII A chain | P00488 | − |
| F13B | Coagulation factor XIII B chain | P05160 | − |
| F9 (FA9) | Coagulation factor IX | P00740 | + |
| FBLN1 | Fibulin 1 | P23142 | − |
| FCN3 | Ficolin-3 | O75636 | − |
| FETUA | Fetuin-A (Alpha-2-HS- | P02765 | − |
| (AHSG) | glycoprotein) | | |
| FETUB | Fetuin-B | Q9UGM5 | + |
| FIBA (FGA) | Fibrinogen alpha chain | P02671 | − |
| FINC (FN1) | Fibronectin 1 | P02751 | − |
| GPX3 | Glutathione peroxidase 3 | P22352 | − |
| HABP2 | Hyaluronan-binding protein 2 | Q14520 | − |
| HBA | Hemoglobin subunit alpha | P69905 | + |
| HBB | Hemoglobin subunit beta | P68871 | + |
| HBD | Hemoglobin subunit delta | P02042 | + |
| HEMO (HPX) | Hemopexin (Beta-1B-glycoprotein) | P02790 | + |
| HEP2 | Heparin cofactor 2 | P05546 | − |
| (SERPIND1) | | | |
| HPT (HP) | Haptoglobin | P00738 | − |
| HPTR (HPR) | Haptoglobin-related protein | P00739 | − |
| IC1 | Plasma protease C1 inhibitor | P05155 | − |
| (SERPING1) | | | |
| IGHA2 | Immunoglobulin Heavy Chain | P01877 | + |
| | Alpha 2 | | |
| IGHG1 | Immunoglobulin Heavy Chain | P01857 | + |
| | Gamma 1 | | |
| IGHG3 | Immunoglobulin Heavy Chain | P01860 | + |
| | Gamma 3 | | |
| IGJ | Immunoglobulin J Chain | P01591 | − |
| ITIH1 | Inter-alpha-trypsin inhibitor H1 | P19827 | − |
| ITIH2 | Inter-alpha-trypsin inhibitor H2 | P19823 | − |
| ITIH4 | Inter-alpha trypsin inhibitor H4 | Q14624 | − |
| KAIN | Kallistatin (Kallikrein inhibitor) | P29622 | − |
| (SERPINA4) | | | |
| KLKB1 | Kallikrein B1 (Plasma kallikrein) | P03952 | − |
| KNG1 | Kininogen-1 | P01042 | − |
| LCAT | Lecithin-cholesterol acyltransferase | P04180 | − |
| LG3BP | Galectin-3-binding protein | Q08380 | + |
| (LGALS3BP) | | | |
| MASP1 | Mannan-binding lectin serine | P48740 | − |
| | protease 1 | | |
| MBL2 | Mannose-binding protein C | P11226 | − |
| PGRP2 | N-acetylmuramoyl-L-alanine | Q96PD5 | − |
| | amidase | | |
| PLF4 (PF4) | Platelet factor 4 (Oncostatin-A, | P02776 | + |
| | CXCL4) | | |
| PLMN (PLG) | Plasminogen | P00747 | + |
| PON1 | Serum paraoxonase/arylesterase 1 | P27169 | − |
| PRG4 (MSF) | Proteoglycan 4 | Q92954 | + |
| PROS | Vitamin K-dependent protein S | P07225 | + |
| SAA4 | Serum amyloid A-4 protein | P35542 | + |
| SEPP1 | Selenoprotein P | P49908 | − |
| (SELP) | | | |
| THBG | Thyroxine-binding globulin | P05543 | − |
| (SERPINA7) | | | |
| THRB (F2) | Prothrombin | P00734 | − |
| TRFE (TF) | Serotransferrin (Transferrin, | P02787 | + |
| | Siderophilin) | | |
| TRY3 (PRSS3) | Trypsin-3 | P35030 | − |
| TSP1 (THBS1) | Thrombospondin-1 | P07996 | + |
| TTHY (TTR) | Transthyretin | P02766 | − |
| VTDB (GC) | Vitamin D-binding protein | P02774 | − |
| VTNC (VTN) | Vitronectin | P04004 | + |
| ZA2G(AZGP1) | Zinc-alpha-2-glycoprotein | P25311 | − |
| ZPI | Protein Z-dependent protease | Q9UK55 | − |
| (SERPINA10) | inhibitor | | |

In some embodiments, the methods of the present disclosure include a step of detecting the level of a panel of microparticle-associated proteins in a biological sample from a pregnant test subject, where the microparticle-associated proteins are from Table 2. In some embodiments, the methods of the present disclosure include a step of detecting the level of at least one microparticle-associated protein in a biological sample from a pregnant test subject, where the at least one protein is selected from Table 2. The proteins listed in Table 2 correspond to proteins with statistically consistent performance as differentiating SPTB from term controls. In some embodiments, the methods of the present disclosure include a step of detecting the level of at least two, at least three, at least four, at least five, at least six, at least seven, at least eight, at least nine, or at least ten microparticle-associated proteins in a biological sample from a pregnant test subject, where the at least one protein is selected from Table 2. In some embodiments, the methods of the present disclosure include a step of detecting the level of five, six, seven, eight, or nine microparticle-associated proteins in a biological sample from a pregnant test subject, where the proteins are selected from Table 2. In an exemplary embodiment, the methods of the present disclosure include a step of detecting the level of five microparticle-associated proteins in a biological sample from a pregnant test subject, where the five proteins are selected from Table 2. In an exemplary embodiment, the methods of the present disclosure include a step of detecting the level of six microparticle-associated proteins in a biological sample from a pregnant test subject, where the six proteins are selected from Table 2. In an exemplary embodiment, the methods of the present disclosure include a step of detecting the level of seven microparticle-associated proteins in a biological sample from a pregnant test subject, where the seventh proteins are selected from Table 2. In an exemplary embodiment, the methods of the present disclosure include a step of detecting the level of eight microparticle-associated proteins in a biological sample from a pregnant test subject, where the eight proteins are selected from Table 2. In an exemplary embodiment, the methods of the present disclosure include a step of detecting the level of nine microparticle-associated proteins in a biological sample from a pregnant test subject, where the nine proteins are selected from Table 2.

TABLE 2
Microparticle-Associated Proteins Differentially
Expressed in Preterm Birth

| Symbol | Protein Name (Alternative Name) | UniProtKB |
|---|---|---|
| A1AG1 | Alpha-1-acid glycoprotein 1 | P02763 |
| (ORM1) | (Orosomucoid-1) | |
| A1AG2 | Alpha-1-acid glycoprotein 2 | P19652 |
| (ORM2) | (Orosomucoid-2) | |
| A1AT | Alpha-1-antitrypsin | P01009 |
| A2AP | Alpha-2-antiplasmin | P08697 |
| (SERPINF2) | | |
| A2GL (LRG) | Leucine-rich alpha-2-glycoprotein | P02750 |
| A2MG (A2M) | Alpha-2-macroglobulin | P01023 |
| ABCF1 | ATP-binding cassette sub-family F | Q8NE71 |
| | member 1 | |
| AFAM | Afamin | P43652 |
| ALBU | Albumin | P02768 |
| ANT3 | Antithrombin-III | P01008 |
| (SERPINC1) | | |
| APOA1 | Apolipoprotein A1 | P02647 |
| APOA4 | Apolipoprotein A1 | P06727 |
| APOC2 | Apolipoprotein C2 | P02655 |
| APOC3 | Apolipoprotein C3 | P02656 |
| APOD | Apolipoprotein D | P05090 |
| APOF | Apolipoprotein F | Q13790 |
| APOL1 | Apolipoprotein L1 | O14791 |
| APOM | Apolipoprotein M | O95445 |
| ATRN | Attractin | O75882 |
| BGH3 | Transforming growth factor-beta induced | Q15582 |
| | protein ig-h3 | |
| BTD | Biotinidase | P43251 |
| C1R | Complement C1r | P00736 |
| C1S | Complement C1s | P09871 |
| C3 | Complement Component 3 | P01024 |
| C4A | Complement Component 4A | P0C0L4 |
| C4BPA | Complement C4b-binding protein alpha | P04003 |
| | chain | |
| C4BPB | C4b-binding protein beta chain | P20851 |
| C7 | Complement Component 7 (CO7) | P10643 |
| C8A | Complement C8 alpha chain | P07357 |
| C8B | Complement Component 8 beta chain | P07358 |
| C9 | Complement C9 (CO9) | P02748 |
| CERU (CP) | Ceruloplasmin (Ferroxidase) | P00450 |
| CFAD (CFD) | Complement Factor D (Adipsin) | P00746 |
| CFAH | Complement Factor H | P08603 |
| CFAI | Complement Factor H | P05156 |
| CXCL7 | Platelet basic protein | P02775 |
| ECM1 | Extracellular matrix protein 1 | Q16610 |
| F10 (FA10) | Coagulation factor X | P00742 |
| F12 (FA12) | Coagulation factor XII | P00748 |
| FBLN1 | Fibulin 1 | P23142 |
| FETUB | Fetuin-B | Q9UGM5 |
| FIBA (FGA) | Fibrinogen alpha chain | P02671 |
| FIBB | Fibrinogen beta chain | P02765 |
| FIBG | Fibrinogen gamma chain | P02679 |
| HABP2 | Hyaluronan-binding protein 2 | Q14520 |
| HBA | Hemoglobin subunit alpha | P69905 |
| HEMO (HPX) | Hemopexin (Beta-1B-glycoprotein) | P02790 |
| HEP2 | Heparin cofactor 2 | P05546 |
| (SERPIND1) | | |
| HPT (HP) | Haptoglobin | P00738 |
| HRG | Histidine-rich glycoprotein | P04196 |
| IC1 | Plasma protease C1 inhibitor | P05155 |
| (SERPING1) | | |
| IGHA1 | Ig alpha-1 chain C region | P01876 |
| IGHA2 | Ig alpha-2 chain C region | P01877 |
| IGHG1 | Immunoglobulin Heavy Chain Gamma 1 | P01857 |
| IGHG2 | Ig gamma-2 chain C region | P01859 |
| IGHG4 | Ig gamma-4 chain C region | P01861 |
| IGHM | Ig mu chain C region | P01871 |
| IPSP | Plasma serine protease inhibitor | P05154 |
| IT1H2 | Inter-alpha-trypsin inhibitor H2 | P19823 |
| ITIH4 | Inter-alpha-trypsin inhibitor heavy | Q14624 |
| | chain H4 | |
| KAIN | Kallistatin (Kallikrein inhibitor) | P29622 |
| (SERPINA4) | | |
| KLKB1 | Kallikrein B1 (Plasma kallikrein) | P03952 |
| KNG1 | Kininogen-1 | P01042 |
| MASP1 | Mannan-binding lectin serine protease 1 | P48740 |
| MBL2 | Mannose-binding protein C | P11226 |
| PEDF | Pigment epithelium-derived factor | P36955 |
| PGRP2 | N-acetylmuramoyl-L-alanine amidase | Q96PD5 |
| PLMN (PLG) | Plasminogen | P00747 |
| PRG4 (MSF) | Proteoglycan 4 | Q92954 |
| SAA4 | Serum amyloid A-4 protein | P35542 |
| SEPP1 (SELP) | Selenoprotein P | P49908 |
| TETN | Tetranectin | P05452 |
| THBG | Thyroxine-binding globulin | P05543 |
| (SERPINA7) | | |
| TRFE (TF) | Serotransferrin (Transferrin, Siderophilin) | P02787 |
| TSP1 (THBS1) | Thrombospondin-1 | P07996 |
| VTDB (GC) | Vitamin D-binding protein | P02774 |
| VTNC (VTN) | Vitronectin | P04004 |
| VWF | Von Willebrand factor | P04275 |
| ZA2G | Zinc-alpha-2-glycoprotein | P25311 |
| (AZGP1) | | |
| ZPI | Protein Z-dependent protease inhibitor | Q9UK55 |
| (SERPINA10) | | |

In another embodiment, the methods of the present disclosure include a step of detecting the level of three proteins selected from the proteins of Table 1, Table 2, Table 4, Table 5, Table 7 or Table 8. In some embodiments, the at least 3 proteins comprise at least HEMO, KLKB1, and TRFE. In some embodiments, the at least 3 proteins comprise at least A2MG, HEMO, and MBL2. In some embodiments, the at least 3 proteins comprise at least KLKB1, IC1, and TRFE. In some embodiments, the at least 3 proteins comprise at least 3 proteins from F13A, IC1, PGRP2, and THBG. In some embodiments, the at least 3 proteins comprise at least IC1, PGRP2, and THBG. In some embodiments, the at least 3 proteins comprise at least CHLE, FETUB, and PROS. In some embodiments, the at least 3 proteins comprise any one of the triplexes presented in Table 7 or Table 8.

In another embodiment, the methods of the present disclosure include a step of detecting the level of at least 3 proteins. In some embodiments, the at least 3 proteins comprise IC1, LCAT, and ITIH4. In some embodiments, the at least 3 proteins can optionally include a fourth protein. In some embodiments the fourth protein is TRFE. In some embodiments, a sample is taken from a pregnant human subject. In some embodiments, the pregnant human subject is primiparous. In some embodiments, the pregnant human subject may have no previous child brought to term. In some embodiments, the pregnant human subject is at 8-14 weeks of gestation, or is at 10-12 weeks of gestation.

In an exemplary embodiment, the methods of the present disclosure include a step of detecting the level of IC1, LCAT, and ITIH4, and the subject is primiparous. In some embodiments, the pregnant human subject is at 8-14 weeks of gestation, or is at 10-12 weeks of gestation.

In an exemplary embodiment, the methods of the present disclosure include a step of detecting the level of IC1, LCAT, TRFE, and ITIH4, and the subject is primiparous. In some embodiments, the pregnant human subject is at 8-14 weeks of gestation, or is at 10-12 weeks of gestation.

In another embodiment, the methods of the present disclosure include a step of detecting the level of at least 4 proteins. In some embodiments, the at least 4 proteins comprise TRFE, IC1, LCAT, and ITIH4. In some embodiments, a sample is taken from a pregnant human subject. In some embodiments, the pregnant human subject is primiparous. In some embodiments, the pregnant human subject may have no previous child brought to term. In some embodiments, the pregnant human subject is at 8-14 weeks of gestation, or is at 10-12 weeks of gestation.

In another embodiment, the methods of the present disclosure include a step of detecting the level of at least 5 proteins. In some embodiments, the at least 5 proteins are F13A, FBLN1, IC1, LCAT, and a fifth protein. In some embodiments, the fifth protein is ITIH1 or ITIH2. In some embodiments, the 5 proteins are F13A, FBLN1, IC1, LCAT, and ITIH1. In some embodiments, the 5 proteins are F13A, FBLN1, IC1, LCAT, and ITIH2. In some embodiments, a sample is taken from a pregnant human subject. In some embodiments, the pregnant human subject is multiparous. In some embodiments, the pregnant human subject is primiparous. In some embodiments, the pregnant human subject is a primigravida. In some embodiments, the pregnant human subject is a multigravida. In some embodiments, the pregnant human subject is at 8-14 weeks of gestation, or is at 10-12 weeks of gestation.

In another embodiment, the methods of the present disclosure include a step of detecting the level of four proteins selected from the proteins of Table 1, Table 2, Table 4, or Table 5. In another embodiment, the methods of the present disclosure include a step of detecting the level of five proteins selected from the proteins of Table 1, Table 2, Table 4, Tor able 5. In another embodiment, the methods of the present disclosure include a step of detecting the level of six proteins selected from the proteins of Table 1, Table 2, Table 4, or Table 5. In another embodiment, the methods of the present disclosure include a step of detecting the level of seven proteins selected from the proteins of Table 1, Table 2, Table 4, or Table 5. In another embodiment, the methods of the present disclosure include a step of detecting the level of eight proteins selected from the proteins of Table 1, Table 2, Table 4, or Table 5.

In another embodiment, the methods of the present disclosure include a step of detecting the level of at least 3, at least 4, at least 5, at least 6, at least 7, or at least 8 proteins selected from the group consisting of FETUB, CBPN, CHLE, C9, F13B, HEMO, IC1, PROS and TRFE.

In another embodiment, the methods of the present disclosure include a step of detecting the level of least 3, at least 4, at least 5, at least 6, at least 7, or at least 8 proteins selected from the group consisting of KLKB1, APOM, ITIH4, IC1, KNG1, C9, APOL1, PGRP2, THBG, FBLN1, ITIH2, VTDB, C8A, APOA1, HPT, and TRY3.

In another embodiment, the methods of the present disclosure include a step of detecting the level of at least 3, at least 4, at least 5, at least 6, at least 7, or at least 8 proteins selected from the group consisting of AACT, KLKB1, APOM, ITIH4, IC1, KNG1, C9, F13B, APOL1, LCAT, PGRP2, FBLN1, ITIH2, CDSL, CBPN, VTDB, AMBP, C8A, ITIH1, TTHY, and APOA1. In some embodiments, at least 3, at least 4, at least 5, at least 6, at least 7, or at least 8 proteins selected from the group consisting of AACT, KLKB1, APOM, ITIH4, IC1, KNG1, C9, F13B, APOL1, LCAT, PGRP2, FBLN1, ITIH2, CDSL, CBPN, VTDB, AMBP, C8A, ITIH1, TTHY, and APOA1 are used to longitudinally monitor a pregnant subject's risk of SPTB. In some embodiments a first sample is taken between 8-14 weeks gestation (e.g. 10-12 weeks) and second sample is taken between 18-24 weeks gestation (e.g. 22-24 weeks). If upon assessment, it is determined that after the second measurement the subject is no longer at risk of SPTB, the management of the remainder of the pregnancy can be adjusted accordingly by a medical professional. Likewise, if upon assessment, it is determined after the second measurement the subject continues to be at risk of SPTB, or is at a greater risk of SPTB than previously determined, the management of the remainder of the pregnancy can be adjusted accordingly by a medical professional.

In another embodiment, the methods of the present disclosure include a step of detecting the level of least 3, at least 4, or at least 5 proteins selected from the group consisting of A1AG1, A2MG, CHLE, IC1, KLKB1, and TRFE.

In another embodiment, the methods of the present disclosure include a step of detecting the level of least 3, at least 4, at least 5, at least 6, at least 7, or at least 8 proteins selected from the group consisting of AACT, A1AG1, A2MG, CBPN, CHLE, C9, F13B, HEMO, IC1, KLKB1, LCAT, PGRP2, PROS, TRFE, A2AP, A2GL, APOL1, APOM, C6, CPN2, FBLN1, ITIH4, KAIN, KNG1, MBL2, SEPP1, THBG, TRY3, AMBP, APOA1, CDSL, C8A, F13A, HPT, ITIH1, and ITIH2.

In another embodiment, the methods of the present disclosure include a step of detecting the level of least 3, at least 4, at least 5, at least 6, at least 7, or at least 8 proteins selected from the group consisting of AACT, A1AG1, A2MG, CBPN, CHLE, C9, F13B, HEMO, IC1, KLKB1, LCAT, PGRP2, PROS, and TRFE.

In another embodiment, the methods of the present disclosure include a step of detecting the level of least 3, at least 4, at least 5, at least 6, at least 7, or at least 8 proteins selected from the group consisting of A2AP, A2GL, APOL1, APOM, C6, CPN2, FBLN1, ITIH4, KAIN, KNG1, MBL2, SEPP1, THBG, and TRY3.

In another embodiment, the methods of the present disclosure include a step of detecting the level of least 3, at least 4, at least 5, at least 6, at least 7, or at least 8 proteins selected from the group consisting of AMBP, APOA1, CDSL, C8A, F13A, HPT, ITIH1, and ITIH2.

Provided herein are panels of microparticle-associated proteins indicative of an increased risk of SPTB. In some embodiments, the panel of microparticle-associated proteins indicative of an increased risk of SPTB comprises at least 3, at least 4, at least 5, at least 6, at least 7, or at least 8 proteins selected from the proteins of Table 1 or Table 2. In some embodiments, the panel of microparticle-associated proteins comprises at least 3, at least 4, at least 5, at least 6, at least 7, or at least 8 proteins selected from the proteins of Table 4. In some embodiments, the panel comprises at least 3, at least 4, at least 5, at least 6, at least 7, or at least 8 proteins selected from the proteins of Table 5. In some embodiments, the panel comprises at least 3 proteins selected from the triplexes of Table 7. In some embodiments, the panel comprises at least 3 proteins selected from the triplexes of Table 8. In some embodiments, the panel comprises at least 3, at least 4, at least 5, at least 6, at least 7, or at least 8proteins selected from the group consisting of FETUB, CBPN, CHLE, C9, F13B, HEMO, IC1, PROS and TRFE. In some embodiments, the panel comprises at least 3, at least 4, at least 5, at least 6, at least 7, or at least 8 proteins selected from the group consisting of KLKB1, APOM, ITIH4, IC1, KNG1, C9, APOL1, PGRP2, THBG, FBLN1, ITIH2, VTDB, C8A, APOA1, HPT, and TRY3. In some embodiments, the panel comprises at least 3, at least 4, at least 5, at least 6, at least 7, or at least 8 proteins selected from the group consisting of AACT, KLKB1, APOM, ITIH4, IC1, KNG1, C9, F13B, APOL1, LCAT, PGRP2, FBLN1, ITIH2, CDSL, CBPN, VTDB, AMBP, C8A, ITIH1, TTHY, and APOA1. In some embodiments, the panel comprises at least 3, at least 4, at least 5 proteins selected from the group consisting of A1AG1, A2MG, CHLE, IC1, KLKB1, and TRFE. In some embodiments, the panel comprises at least 3 proteins selected from the group consisting of F13A, IC1, PGRP2, and THBG. In some embodiments, the panel comprises at least 3, at least 4, at least 5, at least 6, at least 7, or at least 8 proteins selected from the group consisting of AACT, A1AG1, A2MG, CBPN, CHLE, C9, F13B, HEMO, IC1, KLKB1, LCAT, PGRP2, PROS, TRFE, A2AP, A2GL, APOL1, APOM, C6, CPN2, FBLN1, ITIH4, KAIN, KNG1, MBL2, SEPP1, THBG, TRY3, AMBP, APOA1, CDSL, C8A, F13A, HPT, ITIH1, and ITIH2. In some embodiments, the panel comprises at least 3, at least 4, at least 5, at least 6, at least 7, or at least 8 proteins selected from the group consisting of AACT, A1AG1, A2MG, CBPN, CHLE, C9, F13B, HEMO, IC1, KLKB1, LCAT, PGRP2, PROS, and TRFE. In some embodiments, the panel comprises at least 3, at least 4, at least 5, at least 6, at least 7, or at least 8 proteins selected from the group consisting of A2AP, A2GL, APOL1, APOM, C6, CPN2, FBLN1, ITIH4, KAIN, KNG1, MBL2, SEPP1, THBG, and TRY3. In some embodiments, the panel comprises at least 3, at least 4, at least 5, at least 6, or at least 7 proteins selected from the group consisting of AMBP, APOA1, CD5L, C8A, F13A, HPT, ITIH1, and ITIH2. In some embodiments, the panel comprises at least HEMO, KLKB1, and TRFE. In some embodiments, the panel comprises at least A2MG, HEMO, and MBL2. In some embodiments, the panel comprises at least KLKB1, IC1, and TRFE. In some embodiments, the panel comprises at least F13A, IC1, PGRP2, and THBG. In some embodiments, the panel comprises at least IC1, PGRP2, and THBG. In some embodiments, the panel comprises at least CHLE, FETUB, and PROS.

In some embodiments, a first panel (e.g. a first trimester panel, a 8-12 week panel, or a 10-12 week panel) of microparticle-associated proteins indicative of an increased risk of SPTB is provided. In some embodiments, a second panel (e.g. a second trimester panel, a 18-24 week panel, or a 22-24 week panel) of microparticle-associated proteins indicative of an increased risk of SPTB is provided. In some embodiments, a pregnant subject is assessed for risk during the first trimester, between 8-12 weeks gestation or between 10-12 weeks gestation, and then again during the second trimester, 18-24 weeks gestation, or 22-24 weeks gestation. In such embodiments, the useful panel may comprise at least 3, at least 4, at least 5, at least 6, at least 7, or at least 8 proteins from group consisting of AACT, KLKB1, APOM, ITIH4, IC1, KNG1, C9, F13B, APOL1, LCAT, PGRP2, FBLN1, ITIH2, CDSL, CBPN, VTDB, AMBP, C8A, ITIH1, TTHY, and APOA1.

In some embodiments of the panels presented herein, the panel of microparticle-associated proteins indicative of an increased risk of SPTB comprises no more than 30, no more than 25, no more than 20, no more than 15, no more than 10, no more than 9, no more than 8, no more than 7, no more than 6, or no more than 5 microparticle-associated proteins. In an exemplary embodiment, the panel of microparticle-associated proteins indicative of an increased risk of SPTB comprises no more than 5 proteins. In another exemplary embodiment, the panel of microparticle-associated proteins indicative of an increased risk of SPTB comprises no more than 6 proteins. In another exemplary embodiment, the panel of microparticle-associated proteins indicative of an increased risk of SPTB comprises no more than 7 proteins. In another exemplary embodiment, the panel of microparticle-associated proteins indicative of an increased risk of SPTB comprises no more than 8 proteins.

In exemplary embodiments of the panels presented herein, the panel of microparticle-associated proteins indicative of an increased risk of SPTB comprises no more than no more than four or no more than five proteins.

In some embodiments, a first four-biomarker panel (e.g. a first trimester panel, a 8-14 week panel, or a 10-12 week panel) of microparticle-associated proteins indicative of an increased risk of SPTB in primipara subjects is provided. In some embodiments, a second panel (e.g. a second trimester panel, a 18-24 week panel, or a 22-24 week panel) of microparticle-associated proteins indicative of an increased risk of SPTB is provided. In some embodiments, a pregnant subject is assessed for risk during the first trimester, between 8-12 weeks gestation or between 10-12 weeks gestation, and then again during the second trimester, 18-24 weeks gestation, or 22-24 weeks gestation. In such embodiments, the useful panel may comprise at least ICI, ITIH4, TRFE, and LCAT. In such embodiments, the useful panel may consist of ICI, ITIH4, TRFE, and LCAT.

In some embodiments, a first four-biomarker panel (e.g. a first trimester panel, a 8-14 week panel, or a 10-12 week panel) of microparticle-associated proteins indicative of an increased risk of SPTB in primipara or multipara subjects is provided. In some embodiments, a second panel (e.g. a second trimester panel, a 18-24 week panel, or a 22-24 week panel) of microparticle-associated proteins indicative of an increased risk of SPTB is provided. In some embodiments, a pregnant subject is assessed for risk during the first trimester, between 8-12 weeks gestation or between 10-12 weeks gestation, and then again during the second trimester, 18-24 weeks gestation, or 22-24 weeks gestation. In such embodiments, the useful panel may comprise at least F13A, FBLN1, ICI, ITIH1, and LCAT. In such embodiments, the useful panel may consist of F13A, FBLN1, ICI, ITIH1, and LCAT.

In some embodiments, provided herein is a method comprising: preparing a microparticle-enriched fraction from a blood sample from the pregnant subject; and determining a quantitative measure of any one of the panels of microparticle-associated proteins provided herein.

### Pregnant Subjects

The tools and methods provided herein can be used to assess the risk of SPTB in a pregnant subject, wherein the subject can be any mammal, of any species. In some embodiments of the present disclosure, the pregnant subject is a human female. In some embodiments, the pregnant human subject is in the first trimester (e.g., weeks 1-12 of gestation), second trimester (e.g., weeks 13-28 of gestation) or third trimester of pregnancy (e.g., weeks 29-37 of gestation). In some embodiments, the pregnant human subject is in early pregnancy (e.g., from 8, 9, 10, 11, 12, 13, 14, 15, 16, 17, 18, 19 or 20, but earlier than 21 weeks of gestation; from 20, 19, 18, 17, 16, 15, 14, 13, 12, 11, 10 or 9, but later than 8 weeks of gestation). In some embodiments, the pregnant human subject is in mid-pregnancy (e.g., from 21, 22, 23, 24, 25, 26, 27, 28, 29 or 30, but earlier than 31 weeks of gestation; from 30, 29, 28, 27, 26, 25, 24, 23, 22 or 21, but later than 20 weeks of gestation). In some embodiments, the pregnant human subject is in late pregnancy (e.g., from 31, 32, 33, 34, 35, 36 or 37, but earlier than 38 weeks of gestation; from 37, 36, 35, 34, 33, 32 or 31, but later than 30 weeks of gestation). In some embodiments, the pregnant human subject is in less than 17 weeks, less than 16 weeks, less than 15 weeks, less than 14 weeks or less than 13 weeks of gestation; from 20, 19, 18, 17, 16, 15, 14, 13, 12, 11, 10 or 9, but later than 8 weeks of gestation). In some embodiments, the pregnant human subject is in about 8-12 weeks of gestation. In some embodiments, the pregnant human subject is in about 18-14 weeks of gestation. In some embodiments, the pregnant human subject is in about 18-24 weeks of gestation. In an exemplary embodiment, the pregnant human subject is at 10-12 weeks of gestation. In some embodiments, the pregnant human subject is in about 22-24 weeks of gestation. The stage of pregnancy can be calculated from the first day of the last normal menstrual period of the pregnant subject.

Pregnant subjects of the methods described herein can belong to one or more classes or status, including primiparous (no previous child brought to delivery) or multiparous (at least one previous child brought to at least 20 weeks of gestation), primigravida (first pregnancy, first time mother) or multigravida (more than one prior pregnancy). A parity status of primiparous can be denoted as parity of 0 (parity=0); a primiparous status can also be referred to as nulliparous and the terms may be used interchangeably. A parity status of multiparous can be denoted as parity >1 or parity >0, and the terms may be used interchangeably.

In some embodiments, the pregnant human subject is primiparous, i.e. parity=0. In other embodiments, the pregnant subject is multiparous. In some embodiments, the pregnant subject may have brought no previous child to term. In other embodiments, the pregnant subject may have brought at least one previous child to at least 20 weeks of gestation.

In some embodiments, the pregnant human subject is primigravida. In other embodiments, the pregnant subject is multigravida. In some embodiments, the pregnant subject may have had at least one prior SPTB (e.g., birth prior to week 38 of gestation). In some embodiments, the pregnant human subject is asymptomatic. In some embodiments, the subject may have a risk factor of PTB such as a history of pre-gestational hypertension, diabetes mellitus, kidney disease, known thrombophilias and/or other significant preexisting medical condition (e.g., short cervical length).

### Samples

A sample for use in the methods of the present disclosure is a biological sample obtained from a pregnant subject. In preferred embodiments, the sample is collected during a stage of pregnancy described in the preceding section. In some embodiments, the sample is a blood, saliva, tears, sweat, nasal secretions, urine, amniotic fluid or cervicovaginal fluid sample. In some embodiments, the sample is a blood sample, which in preferred embodiments is serum or plasma. In some embodiments, the sample has been stored frozen (e.g., −20° C. or −80° C.).

### Methods for Assessing Risk of Spontaneous Preterm Birth

The phrase “increased risk of spontaneous preterm birth” as used herein indicates that a pregnant subject has a greater likelihood of having a SPTB (before 38 weeks gestation) when one or more preterm birth markers are detected, when a particular panel of microparticle-associated proteins indicative of an increased risk of SPTB are detected, and/or when one or more term birth markers are not detected. In some embodiments, assessing risk of SPTB involves assigning a probability on the risk of preterm birth. In some embodiments, assessing risk of SPTB involves stratifying a pregnant subject as being at high risk, moderate risk, or low risk of SPTB. In some embodiments, assessing risk of SPTB involves determining whether a pregnant subject's risk is increased or decreased, as compared to the population as a whole, or the population in a particular demographic (age, weight, medical history, geography, and/or other factors). In some embodiments, assessing risk of SPTB involves assigning a percentage risk of SPTB.

In some embodiments, the methods provided herein indicate that a pregnant subject has a greater likelihood of having a SPTB between 37 and 38 weeks gestation. In some embodiments, the methods provided herein indicate that a pregnant subject has a greater likelihood of having a SPTB at or before 37 weeks gestation. In some embodiments, the methods provided herein indicate that a pregnant subject has a greater likelihood of having a SPTB at or before 36 weeks gestation. In some embodiments, the methods provided herein indicate that a pregnant subject has a greater likelihood of having a SPTB at or before 35 weeks gestation. In some embodiments, the methods provided herein indicate that a pregnant subject has a greater likelihood of having a SPTB at or before 34 weeks gestation. In some embodiments, the methods provided herein indicate that a pregnant subject has a greater likelihood of having a SPTB at or before 33 weeks gestation. In some embodiments, the methods provided herein indicate that a pregnant subject has a greater likelihood of having a SPTB at or before 32 weeks gestation.

Numerically an increased risk is associated with a hazard ratio of over 1.0, preferably over 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 for preterm birth.

### Detection of Protein Biomarkers

Biomarkers can be detected and quantified by any method known in the art. This includes, without limitation, immunoassay, chromatography, mass spectrometry, electrophoresis and surface plasmon resonance.

In some embodiments, detecting the level (e.g., including detecting the presence) of one or both of SPTB biomarkers and term birth biomarkers is done using an antibody-based method. Suitable antibody-based methods include but are not limited to enzyme linked immunosorbent assay (ELISA), chemiluminescent assay, Western blot, and antibody microarray.

In some embodiments, detecting the level (e.g., including detecting the presence) of one or both of SPTB biomarkers and term birth biomarkers includes detection of an intact protein, or detection of surrogate for the protein, such as a peptide fragment. In some embodiments one or more of the peptide fragments provided in Table 14A are detected (e.g. when the sample is from a pregnant subject who is primiparous). In some embodiments one or more of the peptide fragments provided in Table 14B are detected.

Immunoassay methods include, for example, radioimmunoassay, enzyme-linked immunosorbent assay (ELISA), sandwich assays and Western blot, immunoprecipitation, immunohistochemistry, immunofluorescence, antibody microarray, dot blotting, and FACS.

Chromatographic methods include, for example, affinity chromatography, ion exchange chromatography, size exclusion chromatography/gel filtration chromatography, hydrophobic interaction chromatography and reverse phase chromatography.

In some embodiments, detecting the level of a microparticle-associated protein is accomplished using a mass spectrometry (MS)-based proteomic analysis (e.g. liquid chromatography mass spectrometry LC/MS). In an exemplary embodiment the method involves subjecting a sample to size exclusion chromatography and collecting the high molecular weight fraction (e.g., by size-exclusion chromatography) to obtain a microparticle-enriched sample. The microparticle-enriched sample is then disrupted (using, for example, chaotropic agents, denaturing agents, reducing agents and/or alkylating agents) and the released contents subjected to proteolysis. The disrupted preparation, containing a plurality of peptides.

Proteins in a sample can be detected by mass spectrometry. Mass spectrometers typically include an ion source to ionize analytes, and one or more mass analyzers to determine mass. Ionization methods include, among others, electrospray or laser desorption methods.

Selected reaction monitoring is a mass spectrometry method in which a first mass analyzer selects a polypeptide of interest (precursor), a collision cell fragments the polypeptide into product peptide fragments and one or more of the peptide fragments is detected in a second mass analyzer. When multiple fragments of a polypeptide are analyzed, the method is referred to as Multiple Reaction Monitoring Mass Spectrometry (MRM/MS). Typically, protein samples are digested with a proteolytic enzyme, such as trypsin, to produce peptide fragments. Heavy isotope labeled analogs of certain of these peptides are synthesized as isotopic standards (e.g. Tables 15A and 15B). The isotope-labeled reference peptides (interchangeably referred to herein has isotope standards, stable isotope standard peptides, stable isotopic standards, and SIS) are mixed with a protease-treated sample. The mixture is subjected to mass spectrometry. Peptides corresponding to the daughter ions of the stable isotopic standards (SIS) and the target peptides are detected with high accuracy, in either the time domain or the mass domain. Usually, a plurality of the daughter ions is used to unambiguously identify the presence of a parent ion, and one of the daughter ions, usually the most abundant, is used for quantification. SIS peptides can be synthesized to order, or can be available as commercial kits from vendors such as, for example, e.g., ThermoFisher (Waltham, Mass.) or Biognosys (Zurich, Switzerland).

The assay can include standards that correspond to the analytes of interest (e.g., peptides having the same amino acid sequence as that of analyte peptides), but differ by the inclusion of stable isotopes. Stable isotopic standards can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Accordingly, detection of a protein target by MRM-MS involves detection of one or more peptide fragments of the protein, typically through detection of a stable isotope reference peptide against which the peptide fragment is compared. Typically, an SIS will, itself, be fragmented in a collision cell as will the original digested fragment, and one or more of these fragments is detected by the mass spectrometer.

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5(4):573-588. Mass spectrometry-based assays can be advantageously combined with upstream peptide or protein separation or fractionation methods, such as, for example, with the tandem column system described herein.

In some embodiments, detecting the level (e.g., including detecting the presence) of one or both of SPTB biomarkers and term birth biomarkers is done using a mass spectrometry (MS)-based proteomic analysis, e.g liquid chromatography-mass spectrometry (LC/MS)-based proteomic analysis. In an exemplary embodiment the method involves subjecting a sample to size exclusion chromatography and collecting the high molecular weight fraction to obtain a microparticle-enriched sample. The microparticle-enriched sample is then extracted before digestion with a proteolytic enzyme (e.g. trypsin) to obtain a digested sample comprising a plurality of peptides. The digested sample can then be subjected to a peptide purification/concentration step before liquid chromatography and mass spectrometry to obtain a proteomic profile of the sample. In some embodiments, the purification/concentration step comprises reverse phase chromatography (e.g., ZIPTIP pipette tip with 0.2 μL C18 resin, from Millipore Corporation, Billerica, Mass.).

Table 14A shows exemplary peptides that can be detected to detect an exemplary 4 protein panel of the disclosure (TRFE, IC1, ITIH4, and LCAT) or to detect each protein individually. In some embodiments, the panel is detected using MS/MRM. In some embodiments, the panel is detected using LC-MS/MRM.

In an exemplary embodiment, provided herein is a method for assessing risk of SPTB for a pregnant subject, the method comprising: (a) preparing a microparticle-enriched fraction from a blood sample from the pregnant subject; and (b) determining a quantitative measure of a panel of microparticle-associated proteins in the fraction, wherein the panel comprises ICI, ITIH4, TRFE, and LCAT. In some embodiments, peptides of SEQ ID NO:1, SEQ ID NO:2, SEQ ID NO:3, and SEQ ID NO:4 are detected using MS, MS/MRM, or LC-MS/MRM. In some embodiments, the blood sample is a plasma sample. In some embodiments, the sample is taken from a pregnant subject who is at 8-14 weeks, or 10-12 weeks, or in her first trimester of gestation. In some embodiments the pregnant subject is primiparous. In some embodiments, the pregnant subject is primigravida.

TABLE 14A

| Protein Sequence: | For Detection of: | SEQ ID NO: |
|---|---|---|
| LLDSLPSDTR | IC1 | 1 |
| SSGLVSNAPGVQIR | LCAT | 2 |
| EGYYGYTGAFR | TRFE | 3 |
| ILDDLSPR | ITIH4 | 4 |

Table 14B shows exemplary peptides that can be detected to detect an exemplary 5 protein panel of the disclosure (F13A, FBLN1, ICI, ITIH2, and LCAT), or to detect each protein individually. In some embodiments, the panel is detected using MS/MRM. In some embodiments, the panel is detected using LC-MS/MRM.

In an exemplary embodiment, provided herein is a method for assessing risk of SPTB for a pregnant subject, the method comprising. (a) preparing a microparticle-enriched fraction from a blood sample from the pregnant subject; and (b) determining a quantitative measure of a panel of microparticle-associated proteins in the fraction, wherein the panel comprises F13A, FBLN1, ICI, ITIH1, and LCAT. In some embodiments, peptides of SEQ ID NO:5, SEQ ID NO:6, SEQ ID NO:1, SEQ ID NO:7, and SEQ ID NO:2 are detected using MS, MS/MRM, or LC-MS/MRM. In some embodiments, the blood sample is a plasma sample. In some embodiments, the sample is taken from a pregnant subject who is at 8-14 weeks, or 10-12 weeks, or in her first trimester of gestation. In some embodiments the pregnant subject is primiparous. In some embodiments, the pregnant subject is primigravida. In some embodiments the pregnant subject is multiparous. In some embodiments, the pregnant subject is multigravida.

TABLE 14B

| Protein Sequence | For Detection of: | SEQ ID NO: |
|---|---|---|
| STVLTIPEIIIK | F13A1 | 5 |
| TGYYFDGISR | FBLN1 | 6 |
| LLDSLPSDTR | IC1 | 1 |
| AAISGENAGLVR | ITIH1 | 7 |
| SSGLVSNAPGVQIR | LCAT | 2 |

As provided herein, detection of a biomarker by MS, MS/MRM, or LC-MS/MRM involves detection of one or more peptide fragments of the protein, typically through detection of a stable isotope reference peptide against which the peptide fragment is compared.

Table 15A shows exemplary isotope-labeled reference peptides (isotopic standards) used in the LC-MCS MRM mode for detecting the 4 protein panel (TRFE, IC1, ITIH4, and LCAT) of the disclosure.

In an exemplary embodiment, provided herein is a method for measuring a protein panel, comprising: (a) preparing a microparticle-enriched fraction from a blood sample of a subject; and (b) determining a quantitative measure of a panel of microparticle-associated proteins in the fraction, wherein the panel comprises ICI, ITIH4, TRFE, and LCAT, and wherein the determining comprises measuring surrogate peptides of the proteins. In some embodiments, peptides of SEQ ID NO:1, SEQ ID NO:2, SEQ ID NO:3, and SEQ ID NO:4 are detected, for example using MS, MS/MRM, or LC-MS/MRM. In some embodiments, the method further comprises using the isotope-labeled reference peptides of SEQ ID NO:8 SEQ ID NO:9, SEQ ID NO:10, and SEQ ID NO:11. In some embodiments, the blood sample is a plasma sample. In some embodiments, the sample is taken from a pregnant subject who is at 8-14 weeks, or 10-12 weeks, or in her first trimester of gestation. In some embodiments the pregnant subject is primiparous. In some embodiments, the pregnant subject is primigravida.

In an exemplary embodiment, provided herein is a method for assessing risk of SPTB for a pregnant subject, the method comprising: (a) preparing a microparticle-enriched fraction from a blood sample from the pregnant subject; and (b) determining a quantitative measure of a panel of microparticle-associated proteins in the fraction, wherein the panel comprises ICI, ITIH4, TRFE, and LCAT and wherein the determining comprises measuring surrogate peptides of the proteins. In some embodiments, peptides of SEQ ID NO:1, SEQ ID NO:2, SEQ ID NO:3, and SEQ ID NO:4 are detected using MS, MS/MRM, or LC-MS/MRM and using the isotope-labeled reference peptides of SEQ ID NO:8 SEQ ID NO:9, SEQ ID NO:10, and SEQ ID NO:11. In some embodiments, the blood sample is a plasma sample. In some embodiments, the sample is taken from a pregnant subject who is at 8-14 weeks, or 10-12 weeks, or in her first trimester of gestation. In some embodiments the pregnant subject is primiparous. In some embodiments, the pregnant subject is primigravida.

TABLE 15A
Isotope-Labeled

| Reference | For | |
|---|---|---|
| Peptide (SIS) | Detection of: | SEQ ID NO: |
| LLDSLPSDTR-Isotope | IC1 | 8 |
| SSGLVSNAPGVQIR-Isotope | LCAT | 9 |
| EGYYGYTGAFR-Isotope | TRFE | 10 |
| ILDDLSPR-Isotope | ITIH4 | 11 |

Table 15B shows exemplary isotope-labeled reference peptides (isotopic standards) used in the LC-MCS MRM mode for detecting the 5 protein panel (F13A, FBLN1, ICI, ITIH2, and LCAT) of the disclosure.

In an exemplary embodiment, provided herein is a method for measuring a protein panel, comprising: (a) preparing a microparticle-enriched fraction from a blood sample from a pregnant subject; and (b) determining a quantitative measure of a panel of microparticle-associated proteins in the fraction, wherein the panel comprises F13A, FBLN1, ICI, ITIH1, and LCAT. In some embodiments, peptides of SEQ ID NO:5, SEQ ID NO:6, SEQ ID NO:1, SEQ ID NO:7, and SEQ ID NO:2 are detected using MS, MS/MRM, or LC-MS/MRM. In some embodiments, the method further comprises using the isotope-labeled reference peptides of SEQ ID NO:12, SEQ ID NO:13, SEQ ID NO:8, SEQ ID NO:14, and SEQ ID NO:9. In some embodiments, the blood sample is a plasma sample. In some embodiments, the sample is taken from a pregnant subject who is at 8-14 weeks, or 10-12 weeks, or in her first trimester of gestation. In some embodiments the pregnant subject is primiparous. In some embodiments, the pregnant subject is primigravida. In some embodiments the pregnant subject is multiparous. In some embodiments, the pregnant subject is multigravida.

In an exemplary embodiment, provided herein is a method for assessing risk of SPTB for a pregnant subject, the method comprising: (a) preparing a microparticle-enriched fraction from a blood sample from the pregnant subject; and (b) determining a quantitative measure of a panel of microparticle-associated proteins in the fraction, wherein the panel comprises F13A, FBLN1, ICI, ITIH1, and LCAT. In some embodiments, peptides of SEQ ID N0:5, SEQ ID NO:6, SEQ ID NO:1, SEQ ID NO:7, and SEQ ID NO:2 are detected using MS, MS/MRM, or LC-MS/MRM, and using the isotope-labeled reference peptides of SEQ ID NO:12, SEQ ID NO:13, SEQ ID NO:8, SEQ ID NO:14, and SEQ ID NO:9. In some embodiments, the blood sample is a plasma sample. In some embodiments, the sample is taken from a pregnant subject who is at 8-14 weeks, or 10-12 weeks, or in her first trimester of gestation. In some embodiments the pregnant subject is primiparous. In some embodiments, the pregnant subject is primigravida. In some embodiments the pregnant subject is multiparous. In some embodiments, the pregnant subject is multigravida.

TABLE 15B
Isotope-Labeled

| Reference | For | |
|---|---|---|
| Peptide (SIS) | Detection of: | SEQ ID NO: |
| STVLTIPEIIIK-Isotope | F13A1 | 12 |
| TGYYFDGISR-Isotope | FBLN1 | 13 |
| LLDSLPSDTR-Isotope | IC1 | 8 |
| AAISGENAGLVR-Isotope | ITIH1 | 14 |
| SSGLVSNAPGVQIR-Isotope | LCAT | 9 |

In some embodiments, provided herein are kits comprising a one or more stable isotope reference peptides corresponding to peptide biomarkers, e.g., peptides produced from protease (e.g., trypsin) digestion of biomarker proteins.

In an exemplary embodiment, provided herein is a kit for use in detection of SPTB in a primiparous pregnant subject, wherein the kit comprises the isotope-labeled reference peptides of SEQ ID NO:8, SEQ ID NO:9, SEQ ID NO:10, and SEQ ID NO:11, and instructions for use.

In an exemplary embodiment, provided herein is a kit for use in detection of SPTB in a primiparous or multiparous pregnant subject, wherein the kit comprises the isotope-labeled reference peptides of SEQ ID NO:12, SEQ ID NO:13, SEQ ID NO:8, SEQ ID NO:14, and SEQ ID NO:9, and instructions for use.

In an exemplary embodiment, provided herein is a composition comprising a plurality of protein peptides and a plurality of isotope-labeled reference peptides, wherein the protein peptides comprise, or consist of SEQ ID NO:1, SEQ ID NO:2, SEQ ID NO:3, and SEQ ID NO:4 and the isotope-labeled reference peptides comprise or consist of SEQ ID NO:8, SEQ ID NO:9, SEQ ID NO:10, and SEQ ID NO:11.

In another exemplary embodiment, provided herein is a composition comprising a plurality of protein peptides and a plurality of isotope-labeled reference peptides, wherein the protein peptides comprise, or consist of SEQ ID NO:1, SEQ ID NO:2, SEQ ID NO:5, and SEQ ID NO:6, and SEQ ID NO:7 and the isotope-labeled reference peptides comprise or consist of SEQ ID NO:12, SEQ ID NO:13, SEQ ID NO:8, and SEQ ID NO:14, and SEQ ID NO:9.

In an exemplary embodiment, provided herein is a composition comprising: (i) one or a plurality of peptide fragments of each of one or a plurality of protein biomarkers for preterm birth as disclosed herein and (ii) one or a plurality of isotope-labeled reference peptides (e.g. standard peptides corresponding to SEQ ID N0:8 SEQ ID NO:9, SEQ ID NO:10, and SEQ ID NO:11; or standard peptides corresponding to SEQ ID NO:12, SEQ ID NO:13, SEQ ID NO:8, SEQ ID NO:14, and SEQ ID NO:9) which correspond in amino acid sequence to each of the one or a plurality of peptide fragments, wherein each peptide fragment and isotope-labeled reference peptide has an amino acid sequence corresponding to a peptide fragment produced by protease digestion of the one or a plurality of protein biomarkers. In one embodiment the composition comprises peptide fragments from a microparticle-enriched, protease-digested sample. In another embodiment, one or more of the isotope-labeled reference peptides are selected from Table 15A and 15 B. Further provided are methods (a) comprising providing a sample comprising proteins from a microparticle-enriched fraction of a biological sample; (b) performing protease digestion on the proteins to produce peptide fragments; and (c) contacting the peptide fragments with one or a plurality of isotope-labeled reference peptides ((e.g. standard peptides corresponding to SEQ ID NO:8 SEQ ID NO:9, SEQ ID NO:10, and SEQ ID NO:11; or standard peptides corresponding to SEQ ID NO:12, SEQ ID NO:13, SEQ ID NO:8, SEQ ID NO:14, and SEQ ID NO:9)) corresponding in amino acid sequence to each of the one or a plurality of peptide fragments, wherein each isotope-labeled reference peptide has an amino acid sequence corresponding to a peptide fragment produced by protease digestion of the one or a plurality of protein biomarkers for preterm birth as disclosed herein.

### Classification Algorithms

Methods of assessing risk of SPTB can involve classifying a subject as at increased risk of SPTB based on information including at least a quantitative measure of at least one biomarker of this disclosure. Classifying can employ a classification algorithm or model. Many types of classification algorithms are suitable for this purpose, including linear and non-linear models, e.g., processes such as CART—classification and regression trees), artificial neural networks such as back propagation networks, discriminant analyses (e.g., Bayesian classifier or Fischer analysis), logistic classifiers, and support vector classifiers (e.g., support vector machines). Certain classifiers, such as cut-offs, can be executed by human inspection. Other classifiers, such as multivariate classifiers, can require a computer to execute the classification algorithm.

Classification algorithms can be generated by mathematical analysis, including by machine learning algorithms that perform analysis of datasets of biomarker measurements derived from subjects classed into one or another group. Many machine learning algorithms are known in the art, including those that generate the types of classification algorithms above.

Diagnostic tests are characterized by sensitivity (percentage classified as positive that are true positives) and specificity (percentage classified as negative that are true negatives). The relative sensitivity and specificity of a diagnostic test can involve a trade-off—higher sensitivity can mean lower specificity, while higher specificity can mean lower sensitivity. These relative values can be displayed on a receiver operating characteristic (ROC) curve. The diagnostic power of a set of variables, such as biomarkers, is reflected by the area under the curve (AUC) of an ROC curve.

In some embodiments, the classifiers of this disclosure have a sensitivity of at least 85%, at least 90%, at least 95%, at least 98%, or at least 99%. Classifiers of this disclosure have an AUC of at least 0.6, at least 0.7, at least 0.8, at least 0.9 or at least 0.95.

### Methods for Reducing Risk of Spontaneous Preterm Birth

In one embodiment, if a pregnant subject is determined to be at increased risk of SPTB, the appropriate treatment plans can be employed. By way of example, a surgical intervention such as cervical cerclage and progesterone supplementation have been shown to be effective in preventing preterm birth (Committee on Practice Bulletins, Obstetrics & Gynecology, 120:964-973, 2012). In some embodiments, other measures are taken by health care professionals, such as switching to an at-risk protocol such as increased office visits and/or tracking the patient to a physician specially trained to deal with high risk patients. In some embodiments, if a pregnant subject is determined to be at increased risk of SPTB, steps can be taken such that the pregnant subject will have access to NICU facilities and plans for access to such facilities for rural patients. Additionally, the pregnant subject and family members can have better knowledge of acute-phase symptomatic interventions such as fetal fibronectin testing (diagnostic) and corticosteroids (e.g. for baby lung development) and mag sulfate (e.g. for baby neuroprotective purposes). Additionally, the pregnant subject can be monitored such as better adherence to dietary, smoking cessation, and other recommendations from the physician are followed.

In one embodiment, the pregnant subject is prescribed progesterone supplementation. Currently progesterone supplementation for the prevention of recurrent SPTB is offered to: females with a singleton pregnancy and a prior SPTB; and females with no history of SPTB who have an incidentally detected very short cervix (<15 mm). The present disclosure provides tools to identify additional pregnant subjects that may benefit from progesterone supplementation. These subjects include the following: pregnant females who are primigravidas without a history of risk and without an incidentally detected very short cervix; and pregnant females who are multigravidas but who did not previously have a SPTB.

Pregnant subjects determined to be at increased risk for preterm birth are recommended to receive or are administered progesterone until 36 weeks of gestation (e.g., upon identification or between 16 weeks, 0 days and 20 weeks, 6 days gestation until 36 weeks gestation). In some embodiments, progesterone supplementation comprises 250 mg weekly intramuscular injections. In an exemplary embodiment, the weekly progesterone supplementation comprises administration of hydroxyprogesterone caproate by injection. In other embodiments, progesterone supplementation comprises vaginal progesterone in doses between 50 and 300 mg daily, between 75 and 200 mg daily or between 90 and 110 mg daily.

In another embodiment, in females with a singleton pregnancy determined to be at increased risk for preterm birth and who have had a documented prior SPTB at less than 34 weeks of gestation and short cervical length (less than 25 mm) before 24 weeks of gestation, are recommended to receive or are given a cervical cerclage (also known as tracheloplasty or cervical stitch). In some embodiments, the cervical cerclage is a McDonald cerclage, while in other embodiments it is a Shirodkar cerclage or an abdominal cerclage.

Accordingly, provided herein is one method of decreasing risk of SPTB for a pregnant subject and/or reducing neonatal complications of SPTB, the method comprising: assessing risk of SPTB for a pregnant subject according to any of the methods provided herein; and administering a therapeutic agent, prescribing a revised care management protocol, carrying out fetal fibronectin testing, administering corticosteroids, administering mag sulfate, or increasing the monitoring and surveillance of the subject in an amount effective to decrease the risk of SPTB and/or reduce neonatal complications of SPTB. In some embodiments, the therapeutic agent is selected from the group consisting of a hormone and a corticosteroid. In some embodiments, the therapeutic agent comprises vaginal progesterone or parenteral 17-alpha-hydroxyprogesterone caproate.

### Kits

In another embodiment, a kit of reagents capable of one or both of SPTB biomarkers and term birth biomarkers in a sample is provided. Reagents capable of detecting protein biomarkers include but are not limited to antibodies. Antibodies capable of detecting protein biomarkers are also typically directly or indirectly linked to a molecule such as a fluorophore or an enzyme, which can catalyze a detectable reaction to indicate the binding of the reagents to their respective targets.

In some embodiments, the kits further comprise sample processing materials comprising a high molecular gel filtration composition (e.g., agarose such as SEPHAROSE) in a low volume (e.g., 1 ml) vertical column for rapid preparation of a microparticle-enriched sample from plasma. For instance, the microparticle-enriched sample can be prepared at the point of care before freezing and shipping to an analytical laboratory for further processing, for example by size exclusion chromatography.

In some embodiments, the kits further comprise instructions for assessing risk of SPTB. As used herein, the term “instructions” refers to directions for using the reagents contained in the kit for detecting the presence (including determining the expression level) of a protein(s) of interest in a sample from a subject. The proteins of interest may comprise one or both of SPTB biomarkers and term birth biomarkers. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination.

The invention will be more fully understood by reference to the following examples. They should not, however, be construed as limiting the scope of the invention. It is understood that the examples and embodiments described herein are for illustrative purposes only.

### Examples

Abbreviations: AUC (area under curve); CI (confidence interval); CMP (circulating microparticles); DDN (Differential Dependency Network); FDR (false discovery rate); LC (liquid chromatography); LMP (last menstrual period); MRM (multiple reaction monitoring); MS (mass spectrometry); ROC (receiver operating characteristic); SEC (size exclusion chromatography); SPTB (spontaneous preterm birth); and TERM (full term birth).

### Example 1: Study 1—Identification of SPTB Biomarkers in Samples Obtained Between 10-12 Weeks Gestation

This example describes a study utilizing plasma samples obtained between 10-12 weeks gestation as part of a prospectively collected birth cohort. Singleton cases of SPTB prior to 34 weeks were matched by maternal age, race and gestastional age of sampling to uncomplicated term deliveries after 37 weeks. Circulating microparticles (CMPs) from first trimester samples were isolated and subsequently analyzed by multiple reaction monitoring mass spectrometery (MRM-MS) to identify protein biomarkers. SPTB <34 weeks was assessed given the increased neonatal morbidity in that gestational age range.

### Materials and Methods

Clinical Data and Specimen Collection. Clinical data and maternal K2-EDTA plasma samples (10-12 weeks gestation) were obtained and stored at −80° C. at Brigham and Women's Hospital (BWH), Boston, Mass. between 2009-2014 as part of the prospectively collected LIFECODES birth cohort (McElrath et al., Am J Obstet Gynecol, 207:407-414, 2012). Eligibility criteria included patients who were >18 yrs of age, initiated their prenatal care at <15 weeks of gestation and planned on delivering at the BWH. Exclusion criteria included preexisting medical disorders and fetal anomalies. Gestational age of pregnancy was confirmed by ultrasound scanning <12 weeks gestation. If consistent with last menstrual period (LMP) dating, the LMP was used to determine the due date. If not consistent, then the due date was set by the earliest available ultrasound. Full-term birth was defined as after 37 weeks of gestation, and preterm birth for the purposes of this investigation was defined as SPTB prior to 34 weeks. All cases were independently reviewed and validated by two board certified maternal fetal medicine physicians. When disagreement in pregnancy outcome or characteristic arose, the case was re-reviewed and a consensus conference held to determine the final characterization. Twenty-five singleton cases of SPTB prior to 34 weeks were matched to two control term deliveries by maternal age, race, and gestational age of sampling (plus or minus two weeks).

CMP Enrichment. Plasma samples were shipped on dry ice to the David H Murdock Research Institute (DHMRI, Kannapolis, N.C.) and randomized to blind laboratory personnel performing sample processing and testing to case/control status. CMPs were enriched by size exclusion chromatography (SEC) and isocratically eluted using water (RNAse free, DNAse free, distilled water). Briefly, PD-10 columns (GE Healthcare Life Sciences) were packed with 10 mL of 2% agarose bead standard (pore size 50-150 um) from ABT (Miami, Fla.), washed and stored at 4° C. for a minimum of 24 hrs and no longer than three days prior to use. On the day of use columns were again washed and 1 mL of thawed neat plasma sample was applied to the column. That is, the plasma samples were not filtered, diluted or treated prior to SEC.

The circulating microparticles were captured in the column void volume, partially resolved from the high abundant protein peak (Ezrin et al., Am J Perinatol, 32:605-614, 2015). The samples were processed in batches of 15 to 20 across four days to minimize variability between processing individual samples. One aliquot of the pooled CMP column fraction from each clinical specimen, containing 200 μg of total protein (determined by BCA) was transferred to a 2 mL microcentrifuge tube (VWR) and shipped on dry ice to Biognosys (Zurich, Switzerland) for proteomic analysis.

Liquid Chromatography-Mass Spectrometry. Quantitative proteomic liquid chromatography-mass spectrometry (LC-MS) analysis was performed by Biognosys AG. Briefly, for each sample 20 μg of total protein was lyophilized and then denatured with 8M urea, reduced using dithiothreitol, alkylated with iodoacetamide, and digested overnight with trypsin (Promega). Resulting sample peptides were dried using a SpeedVac system and re-dissolved in 45 μL of Biognosys LC solvent and mixed with Biognosys PlasmaDive (extended version 2.0) stable isotope-labeled reference peptide mix containing Biognosys iRT kit.

Then 1 μg of total protein was injected into an in-house packed C18 column (75 μm inner diameter and 10 cm column length, New Objective); column material was Magic AQ, 3 μm particle size, 200 A pore size from Michrom) on a Thermo Scientific Easy nLC nano-liquid chromatography system. LC-MS-MRM assays were measured on a Thermo Scientific TSQ Vantage triple quadrupole mass spectrometer equipped with a standard nano-electrospray source. The LC gradient for LC-MS-MRM was 5-35% solvent B (97% acetonitrile in water with 0.1% FA) in 30 minutes followed by 35-100% solvent B in 2 minutes and 100% solvent B for 8 minutes (total gradient length was 40 minutes). For quantification of the peptides across samples, the TSQ Vantage was operated in scheduled MRM mode with an acquisition window length of 3.25 minutes. The LC eluent was electrosprayed at 1.9 kV and Q1 was operated at unit resolution (0.7 Da). Signal processing and data analysis was carried out using SpectroDive™ Biognosys' software for multiplexed MRM data analysis based on mProphet (Reiter et al., Nature Methods, 8:430-435, 2011). A Q-value filter of 1% was applied. Protein concentration was determined based on the normalized 1 μg of protein injected into the LC/MS.

Statistical Analysis. To select informative analytes that differentiate SPTB from term deliveries, the processed protein quantitation data were first subjected to univariate receiver-operating characteristic (ROC) curve analysis (Fawcett, Pattern Recognition Letters, 27:861-874, 2006; and Robin et al., BMC Bioinformatics, 19:12:77, 2011). Bootstrap resampling against nulls from sample label permutation was used to control the false-discovery rate (FDR) (Carpenter and Bithell, Statistics in Medicine, 19:1141-1164, 2000; and Xie et al., Bioinformatics, 21:4280-4288, 2005). Briefly, for each protein, a ROC analysis was repeated on bootstrap samples from the original data, the mean and standard deviation (SD) of the area-under-curve (AUC) was estimated. The bootstrap procedure was then applied on the same data again but with sample SPTB status labels randomly permutated. The permutation analysis provided the null results in order to control the FDR and adjust for multiple comparison during the selection of candidate protein biomarkers. The Differential Dependency Network (DDN) bioinformatic tool was then applied in order to extract SPTB phenotype-dependent high-order co-expression patterns among the proteins (Tian et al., Bioinformatics, 32:287-289, 2015). An additional bioinformatic tool, BiNGO, was used to identify gene ontology categories that were overrepresented in the DDN subnetworks in order to explore functional links between the observed proteomic dis-regulations and SPTB (Maere et al., Bioinformatics, 21:3448-3449, 2005). In order to assess the complementary values among the selected proteins and the range of their potential clinically relevant performance, multivariate linear models were derived and evaluated using bootstrap resampling.

### Results

The demographic and clinical characteristics of the sample set are presented in Table 3. Maternal age, race, body mass index (BMI), use of public insurance, smoking during pregnancy, and gestational age at enrollment were similar in both groups. Maternal educational levels were higher in the controls and a greater proportion of the SPTB cases tended to be primiparous.

TABLE 3
Baseline characteristics of SPTB vs.
term control pregnancies, Study 1

| | SPTB (N = 25) | | Controls (N = 50) | | |
|---|---|---|---|---|---|
| | N (%) or | | N (%) or | | |
| Characteristic | Mean (SD) | | Mean (SD) | | p-value^{a} |
| Maternal Age (yrs.) | 32.8 | (7.3) | 31.6 | (5.8) | 0.44 |
| Race | | | | | 0.10 |
| Caucasian | 8 | (32.0%) | 23 | (46.0%) | |
| African-American | 3 | (12.0%) | 5 | (10.0%) | |
| Hispanic | 8 | (32.0%) | 18 | (36.0%) | |
| Asian | 3 | (12.0%) | 2 | (4.0%) | |
| Other | 3 | (12.0%) | 2 | (4.0%) | |
| Maternal BMI (kg/m²) | 29.3 | (6.9) | 27.3 | (7.4) | 0.17 |
| Maternal Education | | | | | 0.004 |
| <High School | 3 | (12.0%) | 0 | (0.0%) | |
| High School/Equivalent | 1 | (4.0%) | 0 | (0.0%) | |
| >High School | 21 | (84.0%) | 50 | (100.0%) | |
| On Public Insurance | 10 | (40.0%) | 14 | (28.0%) | 0.31 |
| Primiparous | 14 | (56.0%) | 15 | (30.0%) | 0.04 |
| Smoked During Pregnancy | 4 | (8.0%) | 1 | (4.0%) | 0.66 |
| Enrollment Gestational age | 11.7 | (3.0) | 11.6 | (3.0) | 0.99 |

^{a}P-values calculated with Wilcoxon Rank Sum test, Chi Square test, Fisher Exact test or ANOVA where appropriate

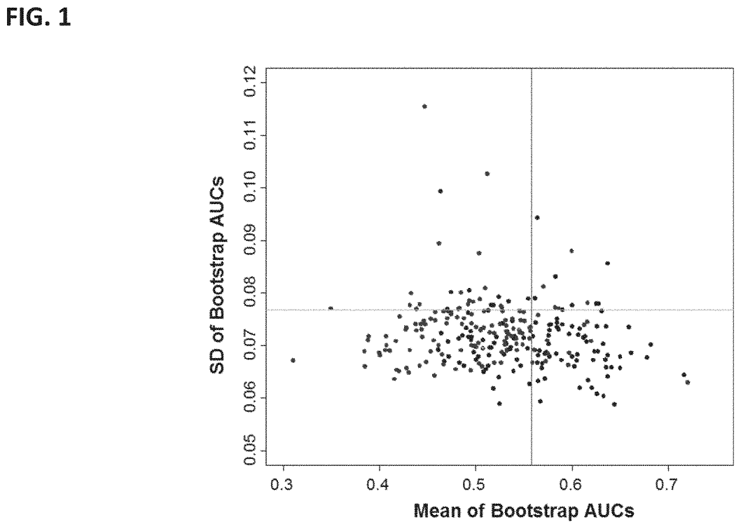

The 132 proteins evaluated via targeted MRM were individually assessed for ability to differentiate SPTB from term deliveries. By requiring that the mean bootstrap AUCs for each candidate protein be significantly greater than the null (>mean+SD of mean bootstrap AUCs estimated with label permutation) and excluding proteins with large bootstrap AUCs variances, 62 of the 132 proteins demonstrated robust power for the detection of SPTB (lower right quadrant of FIG. 1). In contrast, using the same criteria with sample label permutation, only 12 proteins would have been selected. The estimated FDR for protein selection was therefore <20% ( 12/62). These 62 proteins were considered candidates for further multivariate analysis. Table 4 provides performance values for proteins that were downregulated (−) in SPTB cases versus TERM controls, or were upregulated (+) in SPTB cases vs TERM controls. The p value, AUC, and Specificity when Sensitivity is fixed at 65% is shown for biomarkers ranked by AUC from highest to lowest.

TABLE 4
Performance of Single Analytes With Dysregulation

| Names | Direction | AUC | p value | Spec @ Sens 65% |
|---|---|---|---|---|
| AACT | − | 0.715 | 0.003 | 0.740 |
| KLKB1 | − | 0.678 | 0.013 | 0.680 |
| APOM | − | 0.674 | 0.015 | 0.680 |
| ITIH4 | − | 0.662 | 0.024 | 0.660 |
| IC1 | − | 0.651 | 0.034 | 0.460 |
| KNG1 | − | 0.650 | 0.035 | 0.500 |
| TRY3 | − | 0.644 | 0.048 | 0.625 |
| C9 | − | 0.639 | 0.051 | 0.500 |
| F13B | − | 0.635 | 0.058 | 0.580 |
| APOL1 | − | 0.634 | 0.060 | 0.520 |
| LCAT | − | 0.633 | 0.062 | 0.640 |
| PGRP2 | − | 0.631 | 0.067 | 0.600 |
| THBG | − | 0.628 | 0.072 | 0.500 |
| FBLN1 | − | 0.628 | 0.073 | 0.420 |
| ITIH2 | − | 0.628 | 0.073 | 0.540 |
| CD5L | − | 0.627 | 0.075 | 0.580 |
| CBPN | − | 0.626 | 0.077 | 0.520 |
| PROS | + | 0.624 | 0.132 | 0.548 |
| VTDB | − | 0.624 | 0.082 | 0.500 |
| AMBP | − | 0.622 | 0.087 | 0.480 |
| C8A | − | 0.622 | 0.087 | 0.580 |
| ITIH1 | − | 0.622 | 0.089 | 0.520 |
| TTHY | − | 0.619 | 0.095 | 0.480 |
| F13A | − | 0.619 | 0.097 | 0.531 |
| APOA1 | − | 0.618 | 0.100 | 0.540 |
| HPT | − | 0.618 | 0.100 | 0.540 |
| HABP2 | − | 0.615 | 0.107 | 0.520 |
| PON1 | − | 0.612 | 0.118 | 0.600 |
| SEPP1 | − | 0.611 | 0.120 | 0.460 |
| ZA2G | − | 0.610 | 0.125 | 0.540 |
| A2GL | − | 0.607 | 0.134 | 0.520 |
| A2MG | − | 0.606 | 0.139 | 0.440 |
| APOD | − | 0.605 | 0.142 | 0.560 |
| CHLE | − | 0.603 | 0.149 | 0.500 |
| CPN2 | − | 0.603 | 0.149 | 0.480 |
| CLUS | − | 0.602 | 0.152 | 0.400 |
| PLF4 | + | 0.601 | 0.194 | 0.524 |
| THRB | − | 0.597 | 0.176 | 0.420 |
| A1BG | − | 0.590 | 0.206 | 0.560 |
| TRFE | + | 0.590 | 0.206 | 0.540 |
| ZPI | − | 0.585 | 0.241 | 0.420 |
| HEMO | + | 0.583 | 0.247 | 0.440 |
| ATRN | − | 0.582 | 0.249 | 0.480 |
| KAIN | − | 0.580 | 0.263 | 0.500 |
| A1AG1 | − | 0.578 | 0.273 | 0.500 |
| FIBA | − | 0.575 | 0.293 | 0.540 |
| FETUA | − | 0.573 | 0.309 | 0.420 |
| GPX3 | − | 0.571 | 0.320 | 0.531 |
| HEP2 | − | 0.571 | 0.320 | 0.420 |
| FETUB | + | 0.571 | 0.326 | 0.592 |
| C8G | − | 0.570 | 0.325 | 0.480 |
| HPTR | − | 0.570 | 0.325 | 0.400 |
| IGJ | − | 0.568 | 0.342 | 0.460 |
| MBL2 | − | 0.567 | 0.348 | 0.520 |
| C6 | − | 0.567 | 0.348 | 0.440 |
| C1R | − | 0.566 | 0.354 | 0.460 |
| MASP1 | − | 0.563 | 0.378 | 0.440 |
| SAA4 | + | 0.563 | 0.378 | 0.400 |
| FINC | − | 0.562 | 0.390 | 0.400 |
| FCN3 | − | 0.559 | 0.409 | 0.500 |
| A1AG2 | − | 0.556 | 0.435 | 0.480 |
| FA10 | − | 0.556 | 0.435 | 0.340 |
| A1AT | − | 0.554 | 0.455 | 0.400 |
| FA12 | − | 0.551 | 0.488 | 0.362 |
| APOA4 | − | 0.550 | 0.482 | 0.360 |

Individually, 25 of the 62 proteins had the lowest p values (<0.10) and greatest AUC (>0.618) for differentiating SPTB from term controls (Table 5).

TABLE 5
Discriminating Single Analytes

| Protein | p-value | AUC |
|---|---|---|
| AACT | 0.003 | 0.715 |
| KLKB1 | 0.013 | 0.678 |
| APOM | 0.015 | 0.674 |
| ITIH4 | 0.024 | 0.662 |
| IC1 | 0.034 | 0.651 |
| KNG1 | 0.035 | 0.650 |
| TRY3 | 0.048 | 0.644 |
| C9 | 0.051 | 0.639 |
| F13B | 0.058 | 0.635 |
| APOL1 | 0.060 | 0.634 |
| LCAT | 0.062 | 0.633 |
| PGRP2 | 0.067 | 0.631 |
| THBG | 0.072 | 0.628 |
| FBLN1 | 0.073 | 0.628 |
| ITIH2 | 0.073 | 0.628 |
| CD5L | 0.075 | 0.627 |
| CBPN | 0.077 | 0.626 |
| VTDB | 0.082 | 0.624 |
| AMBP | 0.087 | 0.622 |
| C8A | 0.087 | 0.622 |
| ITIH1 | 0.089 | 0.622 |
| TTHY | 0.095 | 0.619 |
| F13A | 0.097 | 0.619 |
| APOA1 | 0.100 | 0.618 |
| HPT | 0.100 | 0.618 |

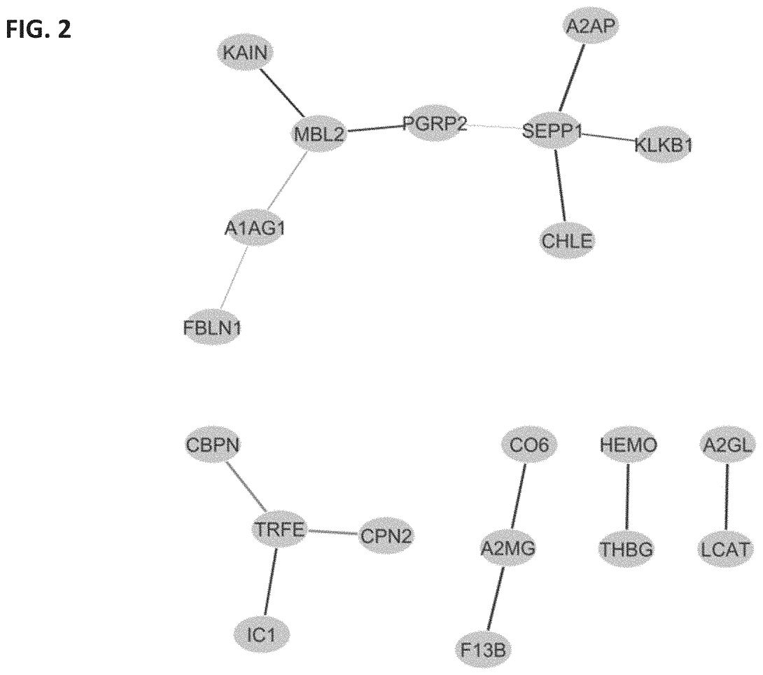

Differential dependency network analysis among the 62 selected proteins identified a number of SPTB phenotype-associated co-expression patterns (FIG. 2). A number of gene ontology categories, such as inflammation, wound healing, the coagulation cascade, and steroid metabolism were overrepresented among the DDN analysis co-expression subnetworks. Table 6 provides a listing of the top discriminating pairwise correlations (p-values <0.001-0.069). There were a total of 20 unique proteins that formed the DDN subnetworks. Several of the pairwise correlations (CBPN-TRFE, CPN2-TRFE, A1AG1-MBL2) were markers for inclusion in the TERM controls rather than the SPTB cases, indicative of protection against SPTB.

TABLE 6
Pair-Wise Connections Between Proteins

| Protein 1 | Protein 2 | Phenotype | p-value |
|---|---|---|---|
| A2AP | SEPP1 | SPTB | <0.001 |
| CBPN | TRFE | TERM | <0.001 |
| CPN2 | TRFE | TERM | <0.001 |
| HEMO | THBG | SPTB | 0.002 |
| A2MG | F13B | SPTB | 0.003 |
| IC1 | TRFE | SPTB | 0.003 |
| KAIN | MBL2 | SPTB | 0.004 |
| A2GL | LCAT | SPTB | 0.005 |
| A2MG | C6 | SPTB | 0.005 |
| CHLE | SEPP1 | SPTB | 0.009 |
| MBL2 | PGRP2 | SPTB | 0.022 |
| KLKB1 | SEPP1 | SPTB | 0.045 |
| A1AG1 | MBL2 | TERM | 0.064 |
| PGRP2 | SEPP1 | SPTB | 0.066 |
| A1AG1 | FBLN1 | SPTB | 0.069 |

Based on the available sample size, and in order to avoid overtraining, only linear models were evaluated to assess the clinically relevant performance and the variables were limited to all possible combinations of two or three proteins out of the 20 proteins in Table 6 (1330 models). Each model was derived and evaluated using 200 bootstrap resampled data in order to estimate the median (90% CI) and specificity for ROC AUCs with a fixed sensitivity of 80%. The top 20 models in terms of the lower-bound of 90% CI of AUCs and specificities are listed in Table 7 and Table 8, respectively. Given limitations imposed by the sample size, the model could not be tested on an independent sample set. To compensate for this the CIs for the panel's performances in the training dataset were estimated through iterative bootstrap analysis. Table 7 shows triplexes from that which, when sensitivity is set at 80%, have the best the area under the curve (AUC). Table 8 shows triplexes from Study 1 which, when sensitivity is set at 80%, have the best specificity.

TABLE 7
Top 20 Models Based on the Lower Bound of 90% CI
of AUC from ROC analysis (SPTB vs. term controls)

| | Specificity at 80% | |
|---|---|---|
| | sensitivity | AUC |
| Panel | Median (90% CI) | Median (90% CI) |
| A2MG HEMO MBL2 | 0.830 (0.654, 0.935) | 0.892 (0.829, 0.949) |
| HEMO IC1 KLKB1 | 0.842 (0.666, 0.927) | 0.892 (0.824, 0.942) |
| A2MG HEMO KLKB1 | 0.812 (0.634, 0.933) | 0.879 (0.819, 0.945) |
| A1AG1 A2MG HEMO | 0.824 (0.666, 0.940) | 0.887 (0.815, 0.943) |
| A1AG1 A2MG C6 | 0.800 (0.630, 0.922) | 0.876 (0.814, 0.932) |
| F13B HEMO KLKB1 | 0.808 (0.643, 0.907) | 0.878 (0.810, 0.931) |
| IC1 KLKB1 TRFE | 0.837 (0.680, 0.939) | 0.882 (0.808, 0.943) |
| HEMO IC1 LCAT | 0.825 (0.653, 0.932) | 0.879 (0.808, 0.938) |
| KLKB1 LCAT TRFE | 0.830 (0.683, 0.935) | 0.870 (0.807, 0.943) |
| A1AG1 KLKB1 TRFE | 0.804 (0.630, 0.919) | 0.876 (0.806, 0.935) |
| A1AG1 HEMO KLKB1 | 0.808 (0.659, 0.918) | 0.872 (0.805, 0.931) |
| A2MG KLKB1 TRFE | 0.811 (0.632, 0.932) | 0.878 (0.804, 0.937) |
| CPN2 HEMO KLKB1 | 0.804 (0.630, 0.922) | 0.871 (0.803, 0.936) |
| A2GL A2MG HEMO | 0.796 (0.543, 0.923) | 0.872 (0.803, 0.933) |
| HEMO KLKB1 PGRP2 | 0.800 (0.637, 0.939) | 0.873 (0.801, 0.932) |
| HEMO KLKB1 LCAT | 0.816 (0.674, 0.940) | 0.874 (0.801, 0.944) |
| A2AP KLKB1 TRFE | 0.821 (0.666, 0.927) | 0.865 (0.800, 0.947) |
| KLKB1 LCAT PGRP2 | 0.808 (0.667, 0.918) | 0.872 (0.798, 0.939) |
| A2MG LCAT TRFE | 0.823 (0.619, 0.928) | 0.871 (0.798, 0.934) |
| A1AG1 HEMO IC1 | 0.802 (0.500, 0.898) | 0.861 (0.797, 0.921) |

TABLE 8
Top 20 Models Based on the Lower Bound of 90% CI of Specificity
at Fixed 80% Sensitivity (SPTB vs. term controls)

| | Specificity at 80% | |
|---|---|---|
| | sensitivity | AUC |
| Panel | Median (90% CI) | Median (90% CI) |
| KLKB1 LCAT TRFE | 0.830 (0.683, 0.935) | 0.870 (0.807, 0.943) |
| IC1 KLKB1 TRFE | 0.837 (0.680, 0.939) | 0.882 (0.808, 0.943) |
| HEMO KLKB1 LCAT | 0.816 (0.674, 0.940) | 0.874 (0.801, 0.944) |
| A2GL KLKB1 TRFE | 0.808 (0.674, 0.920) | 0.865 (0.797, 0.925) |
| KLKB1 LCAT PGRP2 | 0.808 (0.667, 0.918) | 0.872 (0.798, 0.939) |
| HEMO IC1 KLKB1 | 0.842 (0.666, 0.927) | 0.892 (0.824, 0.942) |
| A2AP KLKB1 TRFE | 0.821 (0.666, 0.927) | 0.865 (0.800, 0.947) |
| A1AG1 A2MG HEMO | 0.824 (0.666, 0.940) | 0.887 (0.815, 0.943) |
| A1AG1 HEMO KLKB1 | 0.808 (0.659, 0.918) | 0.872 (0.805, 0.931) |
| A2MG HEMO MBL2 | 0.830 (0.654, 0.935) | 0.892 (0.829, 0.949) |
| HEMO IC1 LCAT | 0.825 (0.653, 0.932) | 0.879 (0.808, 0.938) |
| A2MG HEMO PGRP2 | 0.844 (0.652, 0.961) | 0.874 (0.796, 0.939) |
| F13B HEMO KLKB1 | 0.808 (0.643, 0.907) | 0.878 (0.810, 0.931) |
| KLKB1 PGRP2 TRFE | 0.824 (0.641, 0.915) | 0.876 (0.790, 0.932) |
| HEMO KLKB1 PGRP2 | 0.800 (0.637, 0.939) | 0.873 (0.801, 0.931) |
| A2MG HEMO KLKB1 | 0.812 (0.634, 0.933) | 0.879 (0.819, 0.945) |
| A2AP HEMO PGRP2 | 0.816 (0.633, 0.932) | 0.856 (0.786, 0.926) |
| A2MG KLKB1 TRFE | 0.811 (0.632, 0.932) | 0.878 (0.804, 0.937) |
| CPN2 HEMO KLKB1 | 0.804 (0.630, 0.922) | 0.871 (0.803, 0.936) |
| A1AG1 KLKB1 TRFE | 0.804 (0.630, 0.919) | 0.876 (0.806, 0.935) |

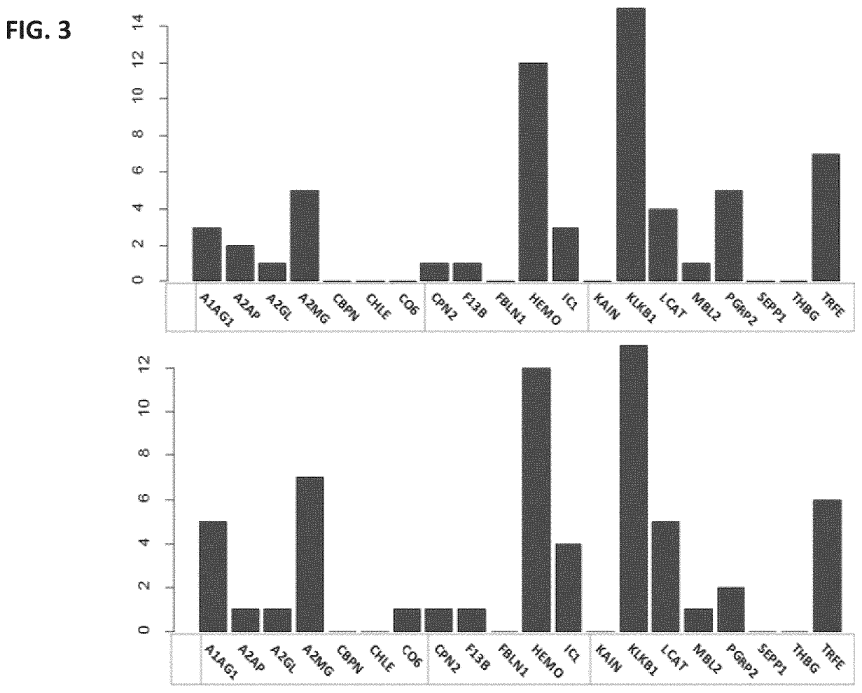

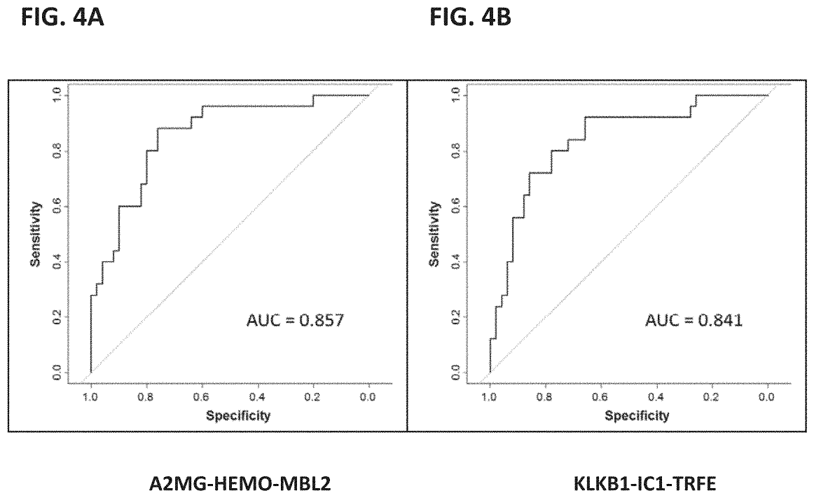

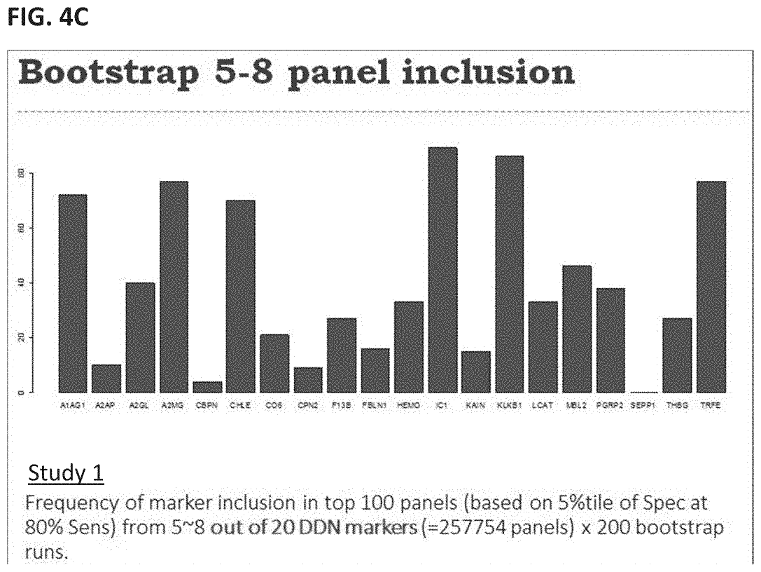

The frequency of individual proteins from the DDN analysis being included in the top 20 model panels was assessed. The protein biomarkers that appeared most frequently were HEMO, KLKB1, and TRFE (FIG. 3). The ROC curve and the AUC was determined by plotting sensitivity and specificity for exemplary linear models using two 3 protein panels (FIG. 4A and FIG. 4B): A2MG, HEMO and MBL2 (FIG. 4A) and KLKB1, IC1, and TRFE (FIG. 4B).

Protein biomarkers with an appreciable single analyte AUC were also selected for evaluation as multiplexing candidates: CBPN, CHLE, C9, F13B, HEMO, IC1, PROS and TRFE. The top 20 five-to-eight marker panels based on AUC and specificity at 75% sensitivity estimated using a linear model and bootstrap resampling.

TABLE 9
Top 20 Five-to-Eight Plex Multimarker Panels

| | Specificity | | | Area Under | | |
|---|---|---|---|---|---|---|
| | at 75% Sensitivity | | | the ROC Curve | | |
| Panel | 5% CI | Median | 95% CI | 5% CI | Median | 95% CI |
| CBPN CHLE C9 F13B | 0.7218 | 0.8857 | 0.9707 | 0.8245 | 0.8947 | 0.9539 |
| HEMO IC1 PROS | | | | | | |
| CBPN CHLE F13B HEMO | 0.7352 | 0.8824 | 0.9730 | 0.8529 | 0.9061 | 0.9601 |
| IC1 PROS TRFE | | | | | | |
| CHLE F13B HEMO IC1 | 0.7564 | 0.8784 | 0.9762 | 0.8430 | 0.9083 | 0.9638 |
| PROS TRFE | | | | | | |
| CBPN CHLE C9 F13B | 0.7273 | 0.8750 | 0.9750 | 0.8363 | 0.9027 | 0.9561 |
| HEMO IC1 PROS TRFE | | | | | | |
| CBPN CHLE F13B IC1 | 0.7218 | 0.8750 | 0.9715 | 0.8291 | 0.8963 | 0.9475 |
| PROS TRFE | | | | | | |
| CHLE C9 F13B HEMO | 0.7419 | 0.8710 | 0.9737 | 0.8505 | 0.9032 | 0.9589 |
| IC1 PROS TRFE | | | | | | |
| CHLE F13B HEMO PROS | 0.7220 | 0.8703 | 0.9668 | 0.8337 | 0.8971 | 0.9484 |
| TRFE | | | | | | |
| CBPN CHLE F13B HEMO | 0.7368 | 0.8697 | 0.9723 | 0.8450 | 0.8960 | 0.9509 |
| IC1 PROS | | | | | | |
| CBPN CHLE C9 F13B | 0.6869 | 0.8675 | 0.9737 | 0.8260 | 0.8986 | 0.9479 |
| HEMO PROS TRFE | | | | | | |
| CHLE C9 F13B HEMO | 0.6998 | 0.8667 | 0.9697 | 0.8269 | 0.8972 | 0.9465 |
| PROS TRFE | | | | | | |
| CBPN CHLE C9 F13B | 0.7185 | 0.8658 | 0.9723 | 0.8124 | 0.8834 | 0.9433 |
| PROS TRFE | | | | | | |
| CBPN CHLE HEMO IC1 | 0.7493 | 0.8658 | 0.9707 | 0.8348 | 0.8946 | 0.9593 |
| PROS TRFE | | | | | | |
| CBPN CHLE C9 F13B | 0.7241 | 0.8649 | 0.9706 | 0.8381 | 0.8971 | 0.9487 |
| IC1 PROS TRFE | | | | | | |
| CBPN CHLE F13B HEMO | 0.6968 | 0.8649 | 0.9677 | 0.8068 | 0.8857 | 0.9422 |
| PROS | | | | | | |
| CHLE F13B IC1 PROS | 0.7199 | 0.8621 | 0.9737 | 0.8315 | 0.9014 | 0.9465 |
| TRFE | | | | | | |
| CBPN CHLE F13B HEMO | 0.7137 | 0.8616 | 0.9586 | 0.8299 | 0.8953 | 0.9523 |
| PROS TRFE | | | | | | |
| CBPN CHLE HEMO IC1 | 0.7218 | 0.8611 | 0.9730 | 0.8183 | 0.8852 | 0.9429 |
| PROS | | | | | | |
| CBPN F13B HEMO IC1 | 0.6995 | 0.8611 | 0.9689 | 0.8102 | 0.8863 | 0.9508 |
| PROS TRFE | | | | | | |
| CHLE C9 F13B IC1 | 0.7212 | 0.8611 | 0.9679 | 0.8212 | 0.8924 | 0.9525 |
| PROS TRFE | | | | | | |
| CHLE C9 F13B HEMO | 0.7239 | 0.8571 | 0.9730 | 0.8368 | 0.8996 | 0.9555 |
| IC1 PROS | | | | | | |

The performance criteria include p-values, specificity at 75% sensitivity, and AUC from ROC analysis. For each criteria, there are three numbers corresponding to bootstrap estimated 95% confidence interval (5% CI, 95% CI) and median (50% CI).

FIG. 4C shows the frequency of marker inclusion in the top 1000 panels (based on 5 percentile of specificity, at 80% sensitivity from five-eight biomarker panels (multiplexes of five to eight proteins) of the 20DDN markers (=257754 panels)×200 bootstrap runs. The six markers that show the highest frequency are A1AG1, A2MG, CHLE, IC1, KLKB1, and TRFE.

### Discussion

Numerous protein biomarkers associated with several clinically relevant biological processes that exhibit characteristic expression profiles by 10-12 weeks gestation among SPTB cases were identified. The protein biomarkers identified are primarily involved in inter-related biological networks linked to coagulation, fibrinolysis, immune modulation and the complement system (Table 10). These systems, in turn, are believed to have an interaction with adaptive immunity and the mediation of inflammatory processes necessary to sustain a successful pregnancy.

TABLE 10
Biological Pathways of CMP-Associated Protein Biomarkers

| Primary Functional | | |
|---|---|---|
| Category | Biomarkers Identified | Additional Biomarkers |
| Coagulation/Wound | F13A, F13B, FBLN1 | FA9, FA10, PROS, |
| Healing | | FIBA, FIBG, FINC, |
| | | HABP2, PLF4 |
| Inflammation/ | CBPN, CHLE, HEMO, | FETUA, FETUB, |
| Oxidative | TRFE, VTDB, PGRP2, | PON1, SAA4, GPX3 |
| Stress | CD5L, SEPP1, CPN2 | |
| Kinin-Kallikrein- | AACT, KLKB1, KNG1, | HEP2 |
| Angiotensin System | KAIN | |
| (coagulation + and | | |
| complement interplay) | | |
| Complement/Adaptive | IC1, C9, CBPN, C6, | C7, ATRN, C1R, |
| Immunity | C8A, HPT, MBL2, | FCN3, HPTR, IGJ, |
| | A2GL, A1AG1 | MASP1, C8G, CLUS, |
| | | A1AG2, A1BG |
| Fibrinolysis/Anti- | ITIH1, ITIH2, ITIH4, | A1AT, ZPI |
| coagulation/ITIH | AMBP, TRY3, A2AP, | |
| Related | A2MG | |
| Lipid Metabolism | APOM, APOL1, | ZA2G, APOD, APOF |
| | APOA1, LCAT | |
| Thyroid Related | THBG, TTHY | THRB |

It is increasingly understood that immune dysregulation, aberrant coagulation and intrauterine inflammation are common to a large proportion of cases of SPTB (Romero et al., Science, 345:760-765, 2014). A high proportion of adverse pregnancy outcomes are believed to have their pathophysiologic origins in early pregnancy. Abnormalities of early placentation and trophoblast function have been observed not only in pregnancies complicated by hypertension, but also in approximately 30% of those experiencing SPTB (Kim et al., Am J Obstet Gynecol, 189:1063-1069, 2003). The state, condition, and function of cells at the maternal-fetal interface during this critical period have already predisposed the pregnancy to adverse outcomes. Others have observed that the concentration of placental-specific microparticles increases significantly with advancing gestation (Sarker et al., J Transl Med, 12:204, 2014). Early perturbations in microparticle-mediated signaling may gradually become magnified as the pregnancy progresses. Ultimately, the anomalies in the maternal fetal cross-talk may become sufficiently great to cause a network crash of the systems that were facilitating tolerance, resulting in a spontaneous preterm birth.

One of the traditional hindrances to a greater understanding of the underlying causes of SPTB is the difficulty of investigating the maternal-fetal interface itself and the unique nature of human placentation. The intrauterine space is both physically and ethically remote. As such, this is perhaps why, with the possible exception of the measurement of cervical length by ultrasound, little recent progress has been made in the development of useful biomarkers to stratify patients according to risk of SPTB (Conde-Agudelo et al., BJOG, 118:1042-1054, 2011). Differences in the protein content of microparticles represent an untapped source of information regarding biology of the maternal-fetal interface. As determined during development of the present disclosure, improved specificity (as indicated by increased AUC) can be obtained with the simultaneous consideration of multiple protein biomarkers associated with a CMP-enriched plasma fraction.

### Example 2: Identification of SPTB Biomarkers in Samples Obtained Between 22-24 Weeks Gestation

This example describes a study utilizing plasma samples obtained between 22-24 weeks gestation, from the same pregnant subjects of Example 1. The sample preparation, analysis and statistical methods were the same as that described for Example 1.

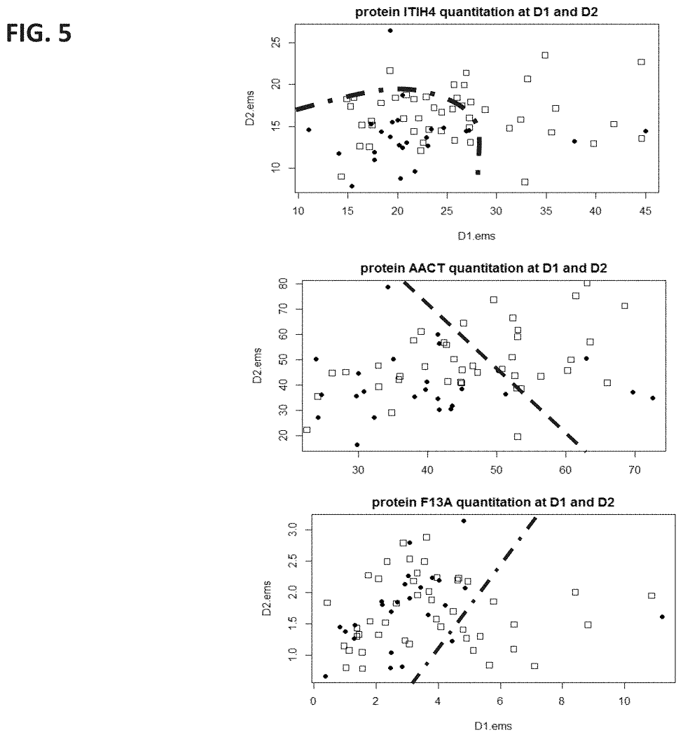

As examples, measurements of three biomarkers (ITIH4, AACT, and F13A) analayzed in Example 1 (time point D1) were plotted against the proteins' corresponding measurements at the later time point of this example (time point D2). This is depicted in FIG. 5—there are different yet clear patterns between D1 and D2 measurements for individual biomarkers that can be used to improve separation between SPTBs and controls. Dash lines indicate possible classification boundaries between SPTB and controls using two time point measurements.

The following proteins displayed consistent performance as predictive for SPTB at week 10-12 (time point D1, Example 1) and week 22-24 (time point D2, this example): AACT, KLKB1, APOM, ITIH4, IC1, KNG1, C9, F13B, APOL1, LCAT, PGRP2, FBLN1, ITIH2, CDSL, CBPN, VTDB, AMBP, C8A, ITIH1, TTHY, and APOA1.

### Example 3: Identification of a Subset of SPTB Biomarkers in Samples Obtained Between 10-12 Weeks Gestation

This example describes a study utilizing plasma samples obtained between 10-12 weeks gestation. Using an independent cohort from that of Example 1, a set of markers was validated that, when obtained between 10-12 weeks, predict SPTB <35 weeks.

### Methods:

Obstetrical outcomes in 75 singleton pregnancies with prospectively collected plasma samples obtained between 10-12 weeks were validated by physician reviewers for SPTB <35 weeks. These were matched to 150 uncomplicated singleton term deliveries. Controls were matched on gestational age at sampling (+/−2 weeks), maternal age (+/−2 years), race and parity. CMPs from these specimens were isolated and analyzed by multiple reaction monitoring mass spectrometry for known protein biomarkers selected from the previous study for their ability to predict the risk of delivery <35 weeks. The biological relevance of these analytes via a combined functional profiling/pathway analysis was also examined.

### Data Analysis and Results:

Cases and controls did not differ by BMI (26 vs 25 kg/m²; p=0.37) or in vitro fertilization (17% vs 10%; p=0.10) status respectively. Mean gestational age at delivery was 33 vs 39 weeks (p<10⁻⁵). It was observed that the CMP markers identified in the previous study again demonstrated distinct Kaplan-Meier curves for SPTB.

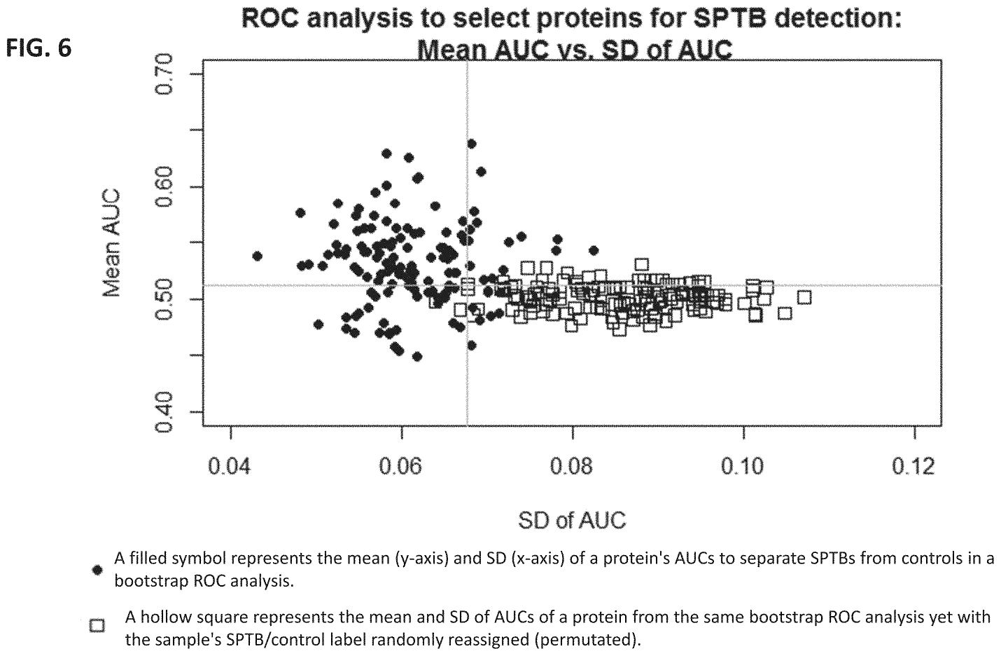

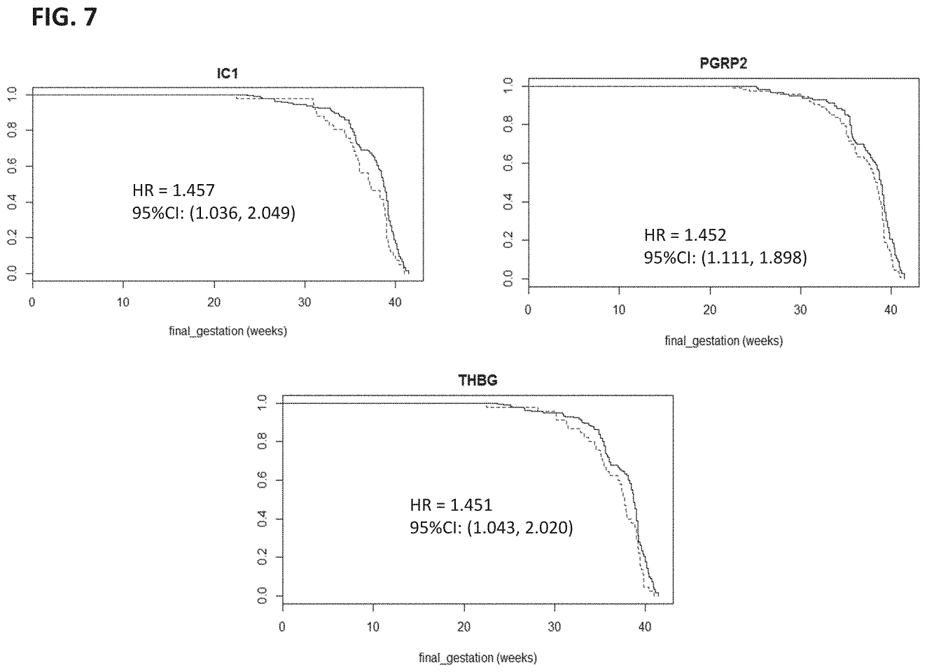

As depicted in FIG. 6, SPTB patients and control samples were randomly sampled with replacement (bootstrap sampling) 50 times. Each time, a receiver-operating characteristic (ROC) curve was computed and the corresponding area-under-curve (AUC) was estimated. The mean (vertical axis) and standard deviation (horizontal axis) of AUCs estimated from the 50 bootstrap sampling runs were plotted for each candidate protein biomarkers (solid filled circles). The same procedure was repeated while the patient/control label of samples were randomly scrambled (label permutation) and the results were plotted as hollow squares, simulating how the results would appear if the protein biomarkers did not have any discriminatory power. The horizontal line indicates one standard deviation above the mean, both estimated from the label permutated results. The vertical line corresponds to one standard deviation above the mean, both estimated from the correctly labeled results. The solid circles in the upper-left quadrant are proteins that had relatively high and statistically stable discriminatory power. Using bootstrap sampling and label permutation analysis, a set of proteins listed in Table 2 above demonstrated statistically consistent differentiating power (as evidenced by ROC analysis) to separate SPTB from controls. A filled symbol represents the mean (y-axis) and SD (x-axis) of a protein's AUCs to separate SPTBs from controls in a bootstrap ROC analysis. A hollow square represents the mean and SD of AUCs of a protein from the same bootstrap ROC analysis yet with the sample's SPTB/control label randomly reassigned (permutated). As shown in FIG. 7, the proteins with statistically consistent performance are presented as filled circles in the upper-left quadrant of the plot.

It was noted that the following proteins displayed consistent performance between the sample set in Example 1 and the sample set in Example 3. These proteins are: KLKB1, APOM, ITIH4, IC1, KNG1, C9, APOL1, PGRP2, THBG, FBLN1, ITIH2, VTDB, CBA, APOA1, HPT, and TRY3.

### Example 4: Sample Preparation Methods

The sample preparation methods were further investigated.

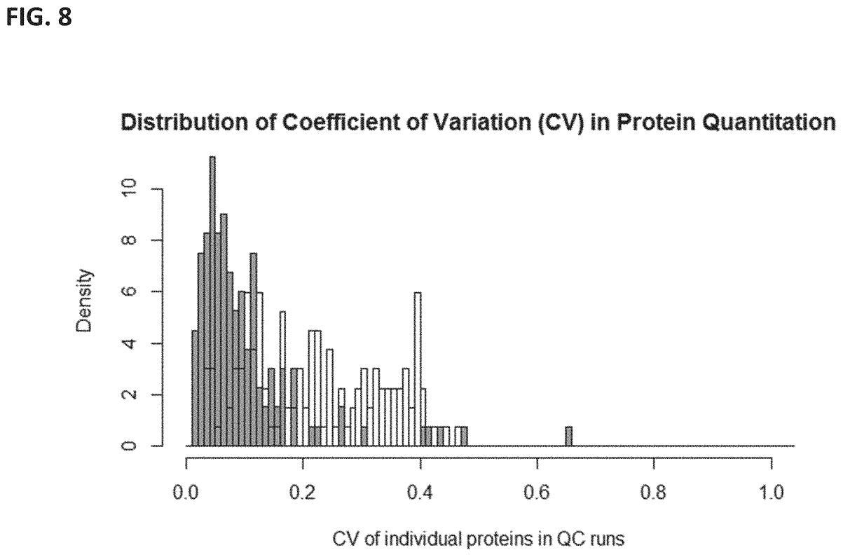

FIG. 8 shows that 2 QC Pools in size exclusion chromatography (SEC) data from samples in Example 2 demonstrate high analytical precision (small coefficient of variation). Two pooled samples were used in sample set used for the data generation of Example 2 (22-24 weeks samples). The coefficient of variation (CV), a measure of analytical precision, was estimated for all proteins using the QC data as technical replicates. The distribution of CVs across all proteins were plotted as histograms. Pool A: shaded bars, Pool B: hollow bars. The analytical precision was proper for biomarker discovery research.

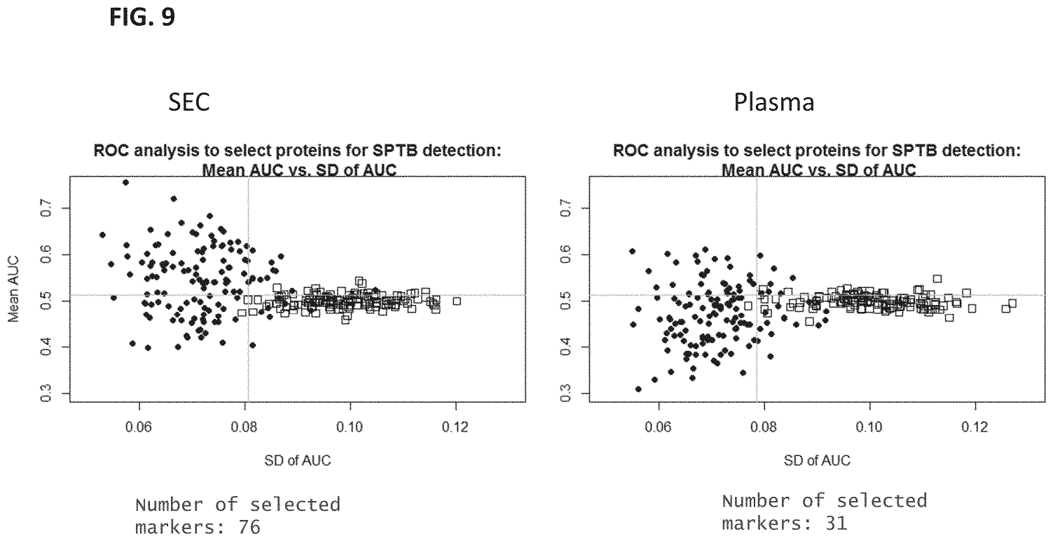

FIG. 9 shows the of NeXosome® sample prep step (SEC) on a number of proteins informative in detecting SPTB from controls, from the 22-24 week samples used in Example 2. The sample bootstrap biomarker selection procedure was applied to data generated from specimens with NeXosome sample preparation step and from plasma specimens directly, both from the same patients. Results show that a large number of informative proteins were identified from data of specimens with SEC. With NeXosome sample prep step (SEC), high value microparticles were enriched, and as a result, improved the identification of clinically informative and biologically relevant biomarkers for SPTB

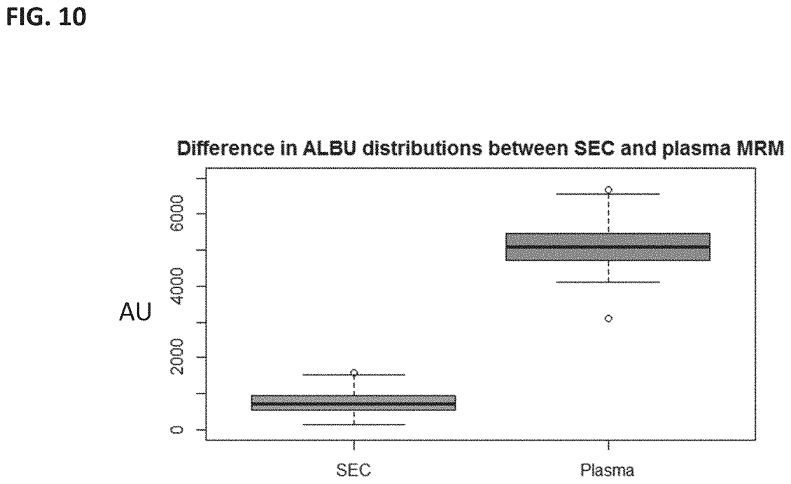

FIG. 10 shows the effect of SEC on concentration of abundant protein albumin (ALBU). Boxplots show distributions of albumin quantitation in samples with SEC prep and in plasma samples directly. The NeXosome sample prep step (SEC) reduced significantly albumin concentration in comparison to using plasma directly.

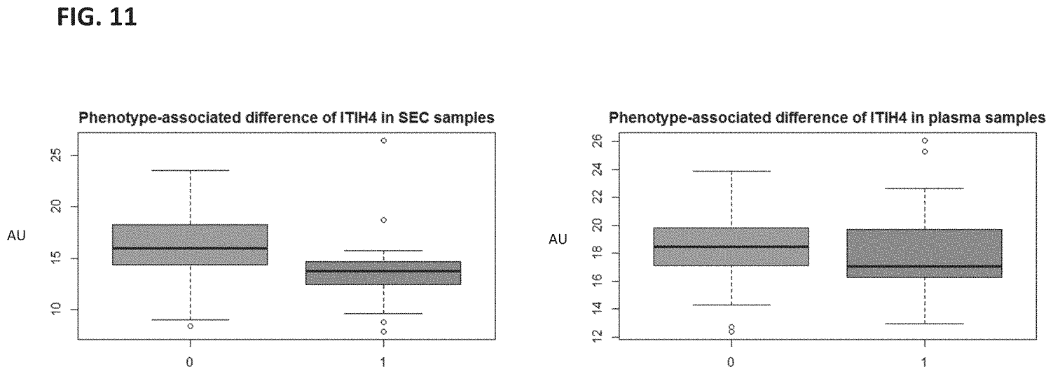

FIG. 11 shows that SEC improved separation between SPTB and controls in D2 ITIH4. Boxplots compare differences in distributions of biomarker ITIH4 between SPTBs and controls in samples with and without NeXosome sample prep step (SEC). SEC significantly improved separation between SPTB and controls for biomarker ITIH4 (p<0.0004 for data from SEC prep samples vs. p=0.3145 for data from plasma directly, Mann-Whitney-Wilcoxon Test).

### Example 5: Study 2—Identification of SPTB Biomarkers in Samples Obtained Between 10-12 Weeks Gestation

This study is a further investigation of the CMP protein multimarker approach in a multicenter population with additional investigation of the testing characteristics by parity and fetal sex.

### Materials and Methods

Clinical Specimen Collection: Maternal EDTA plasma samples (Median 10.2 weeks gestation) were obtained from Brigham and Women's Hospital (BWH), Boston Mass.; the Magee-Women's Research Institute, Pittsburgh Pa.; and, the Global Alliance to Prevent Prematurity and Stillbirth (GAPPS), Seattle Wash. Eligibility criteria included patients who were >18 yrs of age, initiated their prenatal care at <15 weeks of gestation, and planned on delivering at the respective institutions. Exclusion criteria included: preexisting medical disorders (preexisting diabetes, current cancer diagnosis, HIV, and Hepatitis), and fetal anomalies. The analysis was restricted to singleton gestations. Maternal race was determined by self-identification. Gestational age of pregnancy was confirmed by ultrasound scanning at <12 weeks gestation. If consistent with last menstrual period (LMP) dating, the LMP was used to determine the due date. If not consistent, then the due date was set by the earliest available ultrasound <12 weeks gestation. Full-term birth was defined as >37 weeks of gestation, and preterm birth, for the purposes of this example, was defined as sPTB at <35 weeks. The lower limit of the gestational age considered for this analysis was set at 22 weeks. Pregnancies ending <35 weeks gestation were the area of focus for at least two reasons: first, the phenotype of sPTB is generally more homogeneous in this gestational age range and so more likely to be associated with a more uniform set of antecedent pathological processes; and, second, the burden of neonatal morbidity is generally higher in this gestational interval and so it represents a higher-yield target for future prevention.

Patient cases at each center were independently reviewed and validated by physician investigators from the respective centers. The sixty-eight cases of sPTB from Boston, the 9 cases from Magee and the 10 cases from GAPPS were each randomly matched to two control term from the same center. At each center, cases were matched by maternal age (+/−2 years) and gestational age of sampling (+/−2 weeks). The final sample size consisted of 87 cases and 174 controls which included a new collection of 62 cases and 124 controls and 25 cases and 50 controls from a prior analysis (Cantonwine et al. Evaluation of proteomic biomarkers associated with circulating microparticles as an effective means to stratify the risk of SPTB. Am J Obstet Gynecol. 2016; 214(5):631.e1-631.e11). For this example, freshly aliquoted plasma of samples from the cited study were reanalyzed together with the newly acquired samples under a uniform assay protocol in order ensure consistence and minimize potential batch effects. The study protocol was approved by the institutional review boards at each institution, and written, informed consent was obtained from all participating women.

CMP Enrichment: Plasma samples from Magee and GAPPS were shipped on dry ice to BWH and then randomly arranged by laboratory personnel blinded to the case/control status. All 261 samples were then shipped on dry ice to the David H. Murdock Research Institute (DHMRI, Kannapolis, N.C.) where CMPs were enriched by Size Exclusion Chromatography (SEC) and isocratically eluted using the NeXosome Elution Reagent. Briefly, PD-10 columns (GE Healthcare Life Sciences, Pittsburgh, Pa.) were packed with 10 mL of Sepharose 2B Agarose Bead Standard (from a 2% stock solution) purchased from GE Healthcare Bio-Sciences Corporation (Marlborough, Mass.). Columns were washed with Elution Reagent and stored at 4 C° for a minimum of 24 hours and no longer than 3 days prior to use. On the day of use, the columns were again washed with Elution Reagent and 1 mL of thawed plasma sample was applied to the column. The CMPs were captured in the column void volume and resolved from the high abundant protein peak as described (Ezrin A M et al. Circulating serum-derived microparticles provide novel proteomic biomarkers of SPTB. Am J Perinatol. 2015; 32(6):605-14.). To minimize variability between processing, the handling of individual samples was carried out in random batches. An aliquot of the pooled CMP column fraction from each clinical specimen, containing 200 ug of total protein (determined by the BCA reactions), was transferred to a 2 mL microcentrifuge tube (VWR, Radnor, Pa.) and shipped on dry ice to Biognosys (Zurich, Switzerland) for proteomic analysis.

Liquid Chromatography-Mass Spectrometry: Quantitative, proteomic, LC-MS analysis was performed by Biognosys AG. Briefly, for each sample, a total of 20 ug of protein was lyophilized and then denatured with 8M urea, reduced using dithiothreitol, alkylated with the Biognosys alkylation solution, and digested overnight with trypsin (Promega, Madison, Wis.) as previously described. (Ezrin A M et al. Circulating serum-derived microparticles provide novel proteomic biomarkers of SPTB. Am J Perinatol. 2015; 32(6):605-14.) The resulting sample peptides were dried using a SpeedVac system and re-dissolved in 45 uL of the Biognosys LC solvent and mixed and then with the Biognosys PlasmaDive (extended version 2.0) stable isotope-labeled reference peptide mix containing Biognosys iRT kit.

Then 1 μg of total protein was injected into an in-house packed C18 column (75 μm inner diameter and 10 cm column length, New Objective, Woburn, Mass.); the column material was Magic AQ, 3 μm particle size, with a 200 A pore size, from Michrom, Auburn, Calif. This column was used in a Thermo Scientific Easy nLC nano-liquid chromatography system. LC-multiple reaction monitoring (MRM) assays were measured on a Thermo Scientific (Waltham, Mass.) TSQ Vantage, triple quadrupole mass spectrometer equipped with a standard nano-electrospray source. The LC gradient for LC-MRM was a 5-35% gradient of solvent B (97% acetonitrile in water with 0.1% FA), over 30 minutes, followed by 35-100% gradient of solvent B over 2 minutes and then 100% of solvent B for 8 minutes (the total gradient length was 40 minutes).

For quantification of the peptides across samples, the TSQ Vantage was operated in a scheduled MRM mode with an acquisition window length of 3.25 minutes. The LC eluent was electrosprayed at 1.9 kV and the Q1 quadrupole was operated at unit resolution (0.7 Da). Signal processing and data analysis was carried out using SpectroDive™—Biognosys' proprietary software for multiplexed MRM data analysis. A Q-value filter of 1% was applied. Protein concentration was determined based on the normalized 1 ug of protein injected to the LC-MS/MS instrument.

Statistical Analysis: Prior to statistical analysis, the protein quantitation data from the LC-MS/MS MRM assays were normalized into z-scores. The data were then split into a training set and a testing set. The training set consisted of all samples that had been involved in the prior analysis (Cantonwine et al., 2016) as well as 60 samples from the new collection selected through block-randomization. The remaining new collection samples were used as the testing set. The use of block-randomization preserved the case:control ratio in the training and testing sets. The test set was then set aside until step 3 (below) of the analysis.

Univariate analysis (step 1): Within the training set, the candidate set of protein analytes were first subjected to univariate selection for their ability to differentiate sPTB from term deliveries. Briefly, for each protein, receiver-operating-characteristic (ROC) analysis was repeatedly performed 10 times on bootstrapped samples with replacement of the training data. The mean and standard deviation (SD) of the area-under-the-curves (AUCs) from bootstrap ROC analysis were used as measures of the level and statistical stability of the performance, respectively, to rank the putative analytes for their ability to distinguish sPTBs from term deliveries. To establish an objective selection criteria for the analytes and to minimize false discoveries due to random chances, the exactly same bootstrap ROC analysis procedure was applied to the training data set again with the sample labels (i.e., sPTB vs. control) permutated and randomly shuffled. This permutation analysis procedure functionally models the effect of random chance, and serves as a “negative control” in selecting candidate protein markers. Using the same cutoffs on the mean AUCs and SDs, the relative ratio of the number of analytes selected from permutation analysis over that from “real label” analysis allowed for the estimate the false-discovery-rate while controlling for the effect of multiple comparisons.

Multivariate analysis (step 2): The top performing candidate analytes (i.e, with highest mean AUCs and relatively low SDs) from the univariate analysis were then assessed for their complementary values as part of multivariate panels for the prediction of sPTB risk within the training set. To do this, all possible combinations of 5-analyte panels were evaluated using a multivariate classification model with 10 times repeated within-training set cross-validation (each time the model was derived using randomly selected 60% training samples and evaluated on the remaining 40% training samples). Each panel was assessed by three performance metrics: (1) mean AUC, (2) mean sensitivity at a fixed 70% specificity, and (3) mean specificity at a fixed 70% sensitivity, all from within-training cross-validation. The frequencies of individual analytes being a member of the top performing 1% panels of each of the three-performance metrics were then computed. These estimated frequencies served as measures of the ability of the protein analytes to complement one another with regard to differentiating sPTBs from term deliveries and as objective criteria to further reduce the number of candidate biomarkers. The choice of evaluating only 5-analyte panels exhaustively and the use of a particular conservative multivariate model type was based on an exemplary minimally sufficient number of biomarkers to reveal multivariate relationships in analytes for sPTB risk, and a desire not to over-fit the data, as well as the practical constraints of computational complexity. Specifically, the conservative model structure is a support-vector machine (SVM) with radial-basis function kernel. The radius was chosen to be twice of the standard deviations of the analytes. The resulting SVM was therefore heavily constrained and behaved similar to a SVM with linear kernel.

With the number of candidate analytes and their associated panels significantly reduced, the computational approach to fine-tune the parameters of the machine-learning algorithms were used, and afforded an extensive within-training data resampling/cross-validation to finalize and select the top-performing marker panel and associated multivariate predictive model.

Evaluation in the testing set (step3): In the third portion of this analysis, the top performing model was evaluated on the data from the testing set and reported in terms of AUC with associated estimated confidence intervals, sensitivity, and specificity.

Evaluation of parity 0 subset: To evaluate the utility of these analytes in the parity 0 population, the training and testing sets were restricted to primipara mothers (first time mother). The procedures described above were reapplied. Given the sample size restrictions imposed by this stratification, a 4-analyte panel was targeted. As noted, this was to reduce the risk of overfitting the data. In addition to ROC analysis, the 4-analyte model output was used to divide the subjects into high and low risk groups. The two groups were compared using Kaplan-Meier curve by week of gestation. Since the test set represents a case-control sample-set, the purpose of the comparison was meant to graphically demonstrate the noticeable differences in, rather than, the actual shape of the individual Kaplan-Meier curves.

Statistical and model development calculations were carried out in the R 3.2.4 statistical computational environment(17) and using Matlab R2017b (Mathworks, Natick, Mass.).

### Results

The clinical and demographic characteristic of the cases and controls in the entire multicenter cohort are presented in Table 11. Their baseline continuous variables of maternal age, parity, and prepregnancy body mass index (BMI) had similar means. Maternal categorical variables of race, insurance type, smoking, and fetal sex did not differ between cases and controls. Given the design, there were expected differences between gestational age at delivery (p<0.0001) and birthweight (p<0.0001). Importantly, there were no differences between the mean gestational age at sample collection time between the cases or controls.

TABLE 11
Baseline characteristics of SPTB vs. term control pregnancies

| | SPTB (N = 87) | | Controls (N = 174) | | |
|---|---|---|---|---|---|
| | N (%) or | | N (%) or | | p- |
| Characteristic | Mean (SD) | | Mean (SD) | | value^{a} |
| Center | | | | | 0.98 |
| BWH | 68 | (78.2) | 136 | (78.2) | |
| Magee | 9 | (10.3) | 18 | (10.3) | |
| GAPPS | 10 | (11.5) | 20 | (11.5) | |
| Maternal age (yrs.) | 31.2 | (6.2) | 30.7 | (5.6) | 0.66 |
| Race | | | | | 0.82 |
| African-American | 20 | (23.0) | 38 | (21.8) | |
| Not African-American | 67 | (77.0) | 136 | (78.2) | |
| Maternal BMI (kg/m²) | 28.7 | (7.7) | 27.5 | (7.2) | 0.18 |
| Private insurance^{b} | 46 | (67.7) | 97 | (67.8) | 0.54 |
| Maternal smoking during | 9 | (10.3) | 18 | (10.3) | 0.99 |
| pregnancy | | | | | |
| Parity | 1.1 | (1.3) | 1.0 | (1.2) | 0.90 |
| Gestational age at sample | 10.9 | (2.7) | 10.9 | (2.5) | 0.99 |
| collection | | | | | |
| Gestational age at | 31.7 | (3.3) | 39.4 | (1.0) | <0.0001 |
| delivery | | | | | |
| Male fetal sex | 42 | (48.8) | 86 | (49.1) | 0.96 |

^{a}P-values calculated with Wilcoxon Rank Sum test, Chi Square test, Fisher Exact test or ANOVA where appropriate
^{b}N = 59 missing

The total sample set of 261 was split randomly into training and testing sets. Forty-five cases of sPTB and 90 term controls comprised the training set and the remaining 42 cases of sPTB and 84 term controls made up the testing set. The characteristics of the new training and testing sets are compared in Table 12.

TABLE 12
Characteristics of the secondary validation and training set

| | Training set | | | | | Test set | | | | | |
|---|---|---|---|---|---|---|---|---|---|---|---|
| | SPTB | | Control | | | SPTB | | Control | | | p-value^{a} |
| | (N = 45) | | (N = 90) | | p-value^{a} | (N = 42) | | (N = 84) | | p-value^{a} | (SPTB in |
| | Mean (SD) | | Mean (SD) | | (SPTB vs. | Mean (SD) | | Mean (SD) | | (SPTB vs. | training vs. |
| Variable | or N (%) | | or N (%) | | control) | or N (%) | | or N (%) | | control) | validation) |
| Maternal | 32.4 | (6.6) | 31.5 | (5.6) | 0.49 | 30.0 | (5.6) | 29.9 | (5.6) | 0.93 | 0.09 |
| age (yrs) | | | | | | | | | | | |
| Race | | | | | | | | | | | |
| African | 7 | (15.6%) | 13 | (14.4%) | 0.86 | 13 | (30.9%) | 25 | (29.4%) | 0.86 | 0.09 |
| American | | | | | | | | | | | |
| Not African | 38 | (84.4%) | 77 | (85.6%) | | 29 | (69.1%) | 59 | (70.6%) | | |
| American | | | | | | | | | | | |
| Prepregnancy | 29.3 | (7.7) | 27.4 | (6.9) | 0.16 | 28.0 | (7.7) | 27.5 | (7.6) | 0.67 | 0.41 |
| BMI | | | | | | | | | | | |
| Parity | 1.2 | (1.4) | 1.1 | (1.9) | 0.74 | 1.0 | (1.3) | 1.1 | (1.2) | 0.86 | 0.62 |
| Smoked in | 3 | (6.7%) | 8 | (8.9%) | 0.75 | 6 | (14.3%) | 10 | (11.8%) | 0.69 | 0.30 |
| pregnancy | | | | | | | | | | | |
| Past history | 12 | (26.7%) | 7 | (7.8%) | 0.007 | 19 | (45.2%) | 35 | (41.2%) | 0.71 | 0.08 |
| of PTB | | | | | | | | | | | |
| Gestational | 4 | (8.9%) | 4 | (4.4%) | 0.44 | 2 | (4.8%) | 7 | (8.2%) | 0.47 | 0.68 |
| Diabetes | | | | | | | | | | | |
| Male fetus | 23 | (51.1%) | 46 | (51.1%) | 0.99 | 19 | (46.3) | 40 | (47.1%) | 0.94 | 0.67 |
| Birthweight | 1889 | (679) | 3488 | (467) | <0.0001 | 1656 | (611) | 3318 | (467) | <0.0001 | 0.24 |
| Gestational | 11.0 | (2.8) | 11.1 | (2.6) | 0.78 | 10.9 | (2.6) | 10.7 | (2.4) | 0.71 | 0.89 |
| at sampling | | | | | | | | | | | |

^{a}P-values calculated with Wilcoxon Rank Sum test, Chi Square test, or Fisher Exact test where appropriate

An initial inclusion of 36 protein analytes was based on discriminatory performance in the prior analysis. (Cantonwine et al., 2016). The 35 protein analytes targeted for quantification are identified in Table 13 below.

TABLE 13
Microparticle-Associated peptides quantified

| Symbol | Protein Name (Alternative Name) | UniProtKB |
|---|---|---|
| A1AG1 (ORM1) | Alpha-1-acid glycoprotein 1 | P02763 |
| | (Orosomucoid-1) | |
| A2AP | Alpha-2-antiplasmin | P08697 |
| (SERPINF2) | | |
| A2GL (LRG) | Leucine-rich alpha-2-glycoprotein | P02750 |
| A2MG (A2M) | Alpha-2-macroglobulin | P01023 |
| AACT | Alpha-1-antichymotrypsin | P01011 |
| (SERPINA3) | | |
| AMBP | Alpha-1-microglobulin/bikunin | P02760 |
| | precursor | |
| APOA1 | Apolipoprotein A1 | P02647 |
| APOL1 | Apolipoprotein L1 | O14791 |
| APOM | Apolipoprotein M | O95445 |
| CPN1 (CBPN) | Carboxypeptidase N, polypeptide 1 | P15169 |
| CD5L | CD5 antigen-like | O43866 |
| C6 | Complement C6 (CO6) | P13671 |
| C8A | Complement C8 alpha chain (CO8A) | P07357 |
| C9 | Complement C9 (CO9) | P02748 |
| CPN2 | Carboxypeptidase N, polypeptide 2 | P22792 |
| F13A | Coagulation factor XIII A chain | P00488 |
| F13B | Coagulation factor XIIIB chain | P05160 |
| FBLN1 | Fibulin 1 | P23142 |
| HEMO (HPX) | Hemopexin (Beta-1B-glycoprotein) | P02790 |
| HPT (HP) | Haptoglobin | P00738 |
| IC1 (SERPING1) | Plasma protease C1 inhibitor | P05155 |
| ITIH1 | Inter-alpha-trypsin inhibitor H1 | P19827 |
| ITIH2 | Inter-alpha-trypsin inhibitor H2 | P19823 |
| ITIH4 | Inter-alpha trypsin inhibitor H4 | Q14624 |
| KLKB1 | Kallikrein B1 (Plasma kallikrein) | P03952 |
| KNG1 | Kininogen-1 | P01042 |
| LCAT | Lecithin-cholesterol acyltransferase | P04180 |
| LG3BP | Galectin-3-binding protein | Q08380 |
| (LGALS3BP) | | |
| MBL2 | Mannose-binding protein C | P11226 |
| PGRP2 | N-acetylmuramoyl-L-alanine amidase | Q96PD5 |
| SEPP1 (SELP) | Selenoprotein P | P49908 |
| THBG | Thyroxine-binding globulin | P05543 |
| (SERPINA7) | | |
| TRFE (TF) | Serotransferrin (Transferrin, | P02787 |
| | Siderophilin) | |
| TRY3 (PRSS3) | Trypsin-3 | P35030 |
| TTHY (TTR) | Transthyretin | P02766 |
| VTDB (GC) | Vitamin D-binding protein | P02774 |

Within the training set, these 36 analytes were further sub-selected as described above through multivariate analysis for their complementary contribution in top-performing panels. FIG. 13 displays the frequency with which individual analytes were members of the top 1% of performing panels with respect to ROC-AUC analysis among all possible 376,992 combinations of 5-analyte panels, with specificities determined at a fixed sensitivity of 70%, and sensitivities determined at a fixed specificity of 70%. Based on the results, panels of eligible analytes were cross-validated to form final panels. Taken as individual markers, the CMP-associated proteins encompassing F13A, FBLN1, IC1, ITIH2 and LCAT yielded the most stable performance based on repeated cross-validation evaluation within the training data. The AUC is shown as a dark gray bar, specificity at fixed specificity at 70% is shown as a black bar, and sensitivity at fixed sensitivity at 70% is shown as a light gray bar. The models were run by fixing either sensitivity or specificity, and determining which marker combinations were optimal for the panel performance in those cases. These data support the selection of the above 5-protein panel, without regarding for parity status or other factors.

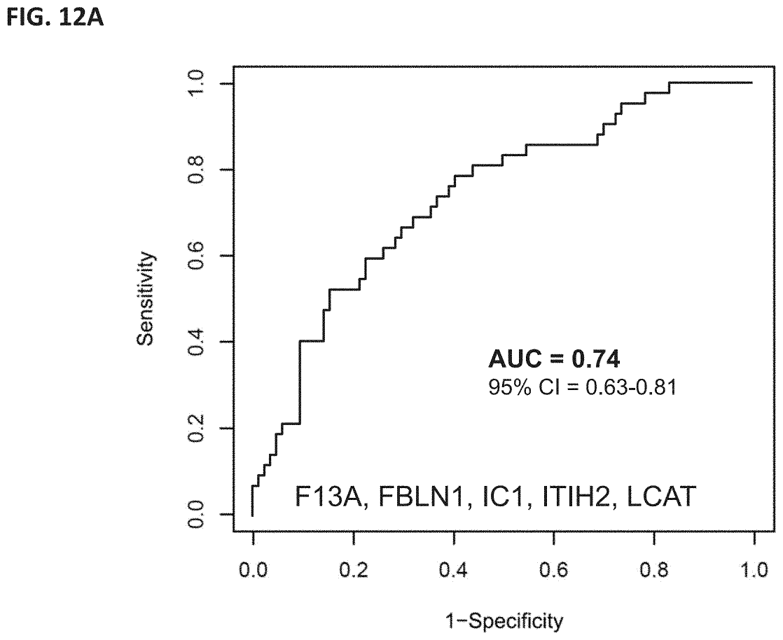

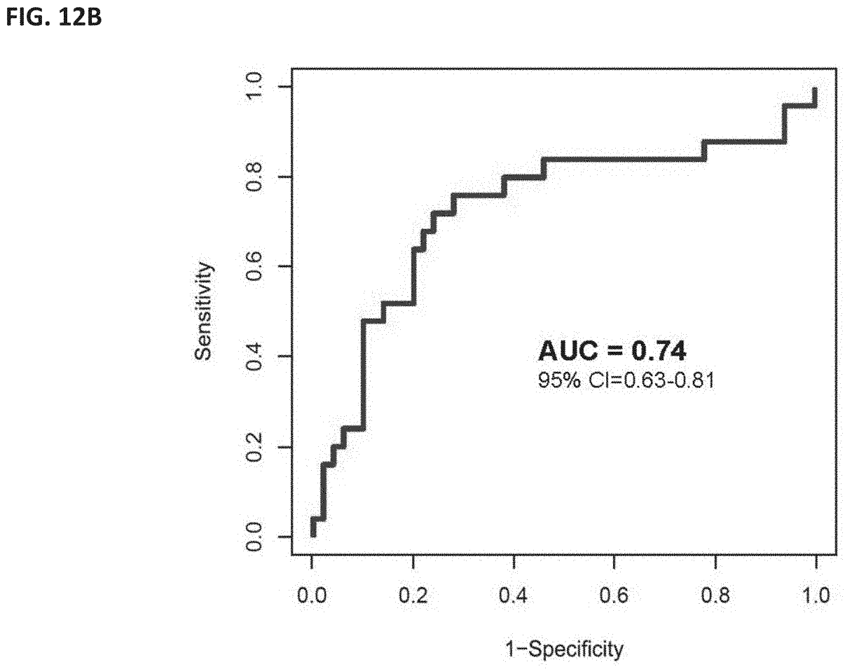

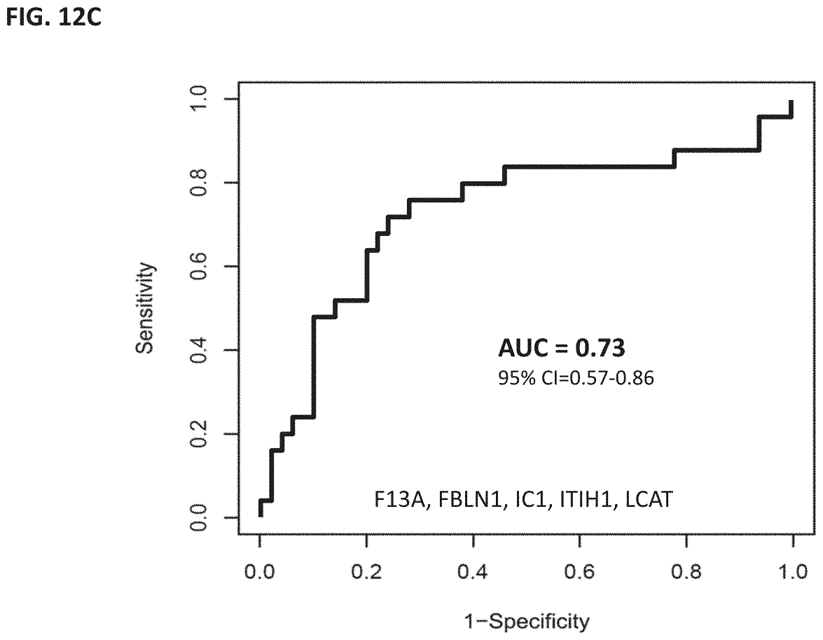

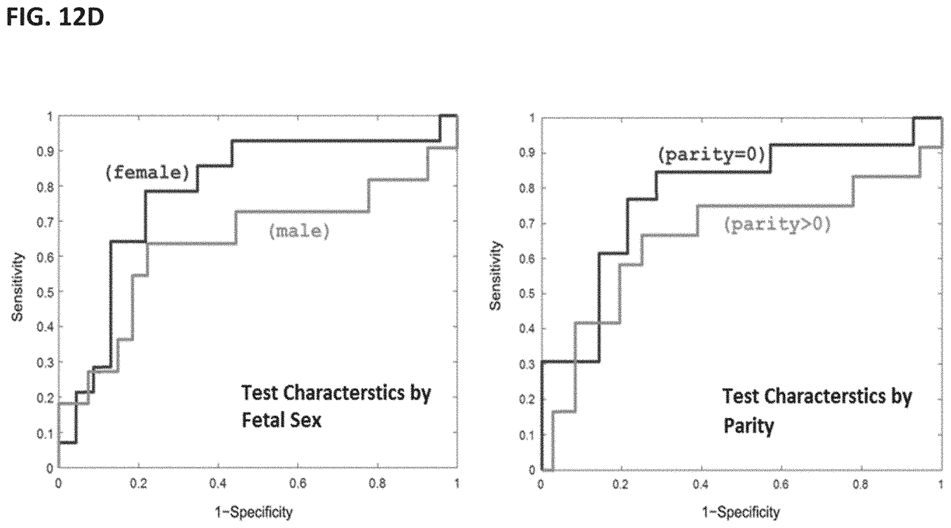

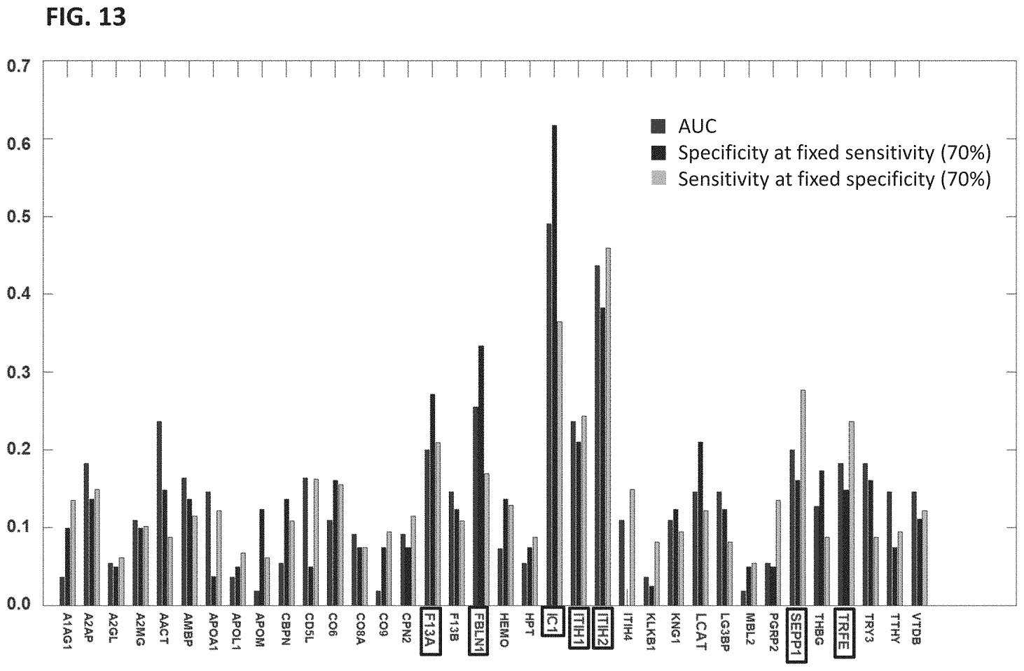

Combining these individual markers and applying them to the test data as a multi-marker panel, the combination of F13A, FBLN1, IC1, ITIH2, and LCAT demonstrated an AUC of 0.74 (95% CI 0.63-0.81) from ROC analysis (FIG. 12A and FIG. 12B). A cutoff of the score maximizing both sensitivity and specificity yields values of 0.70 and 0.81 respectively. The positive likelihood ratio would be 2.70 with a negative likelihood ratio of 0.27. Assuming a hypothetical population of 1000, the 95% confidence intervals would be, respectively, 2.29-3.19 and 0.15-0.48. Test performance did not change with body mass index. This 5-protein marker panel was optimized for use in all subjects regardless of parity status or other factors such as fetal gender.

FIG. 12C presents the ROC for a 5 protein panel including F13A, FBLN1, IC1, ITIH1, and LCAT with an associated AUC of 0.73 (95% CI: 0.57-0.86). Test performance did not change with body mass index. This 5 protein marker panel was also optimized for use in all subjects regardless of parity status or other factors such as fetal gender. FIG. 12D demonstrates that test performance was increased for female (AUC 0.79) vs. male fetuses (AUC 0.64) and for nulliparous (parity=0) (AUC 0.78) as opposed to multiparous (parity >1) (AUC 0.66).

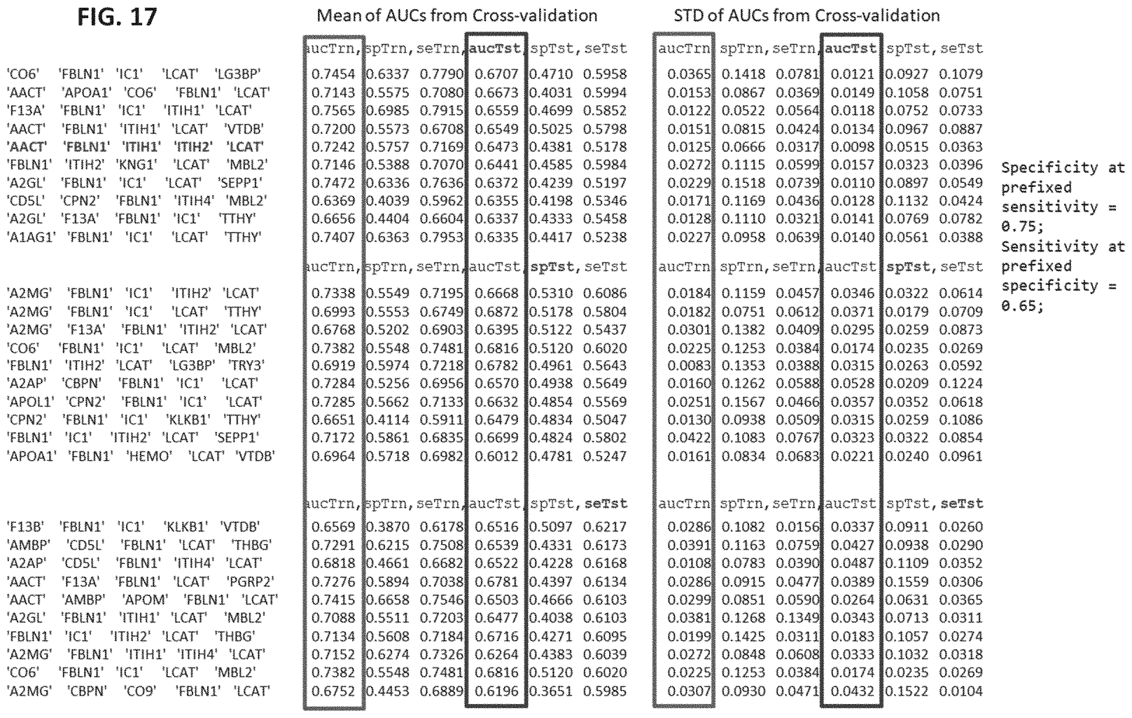

FIG. 17 shows other 5-marker panels and their training/cross-validation performance of some of the top performing panels in terms of mean and standard deviation of AUC, with the sensitivity at a prefixed specificity (0.65) and specificity at prefixed sensitivity (0.75).

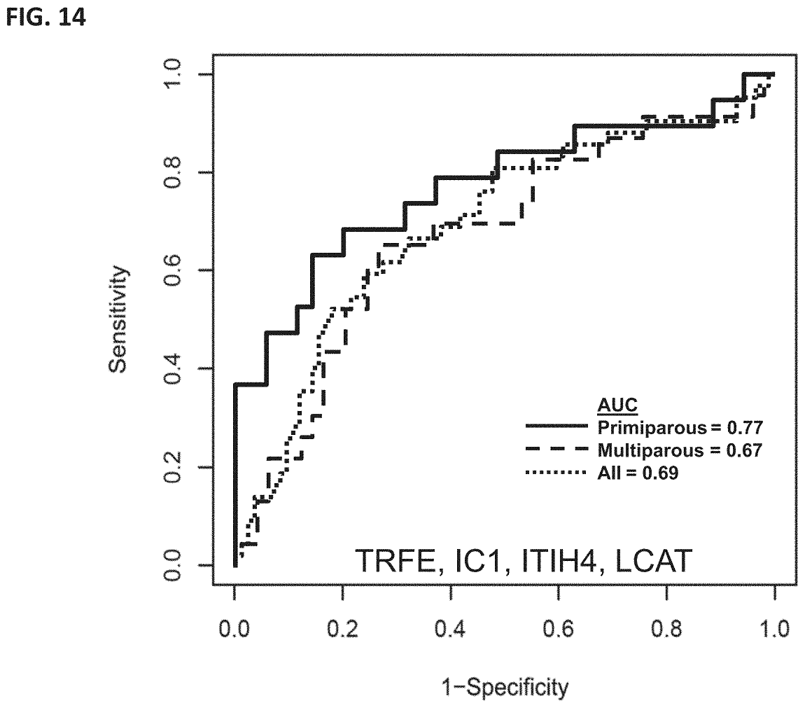

The same work flow was again used on the training set but now with the purpose of selecting analyte combinations to discern the risk of sPTB only among primipara mothers. The process described above, through cross-validation, on the training set resulted in the combination of the TRFE, IC1, ITIH4 and LCAT proteins as the highest performing multi-marker panel classifying primipara mothers. In the testing data, this 4-plex combination demonstrated an AUC of 0.77 (95% CI: 0.61-0.90), as displayed in FIG. 14. At a specificity of 0.86, the corresponding sensitivity would be 0.63. The positive likelihood ratio would be 4.50, with a negative likelihood ratio of 0.43. Assuming a hypothetical population of 1000, the 95% confidence intervals would be, respectively, 3.45-5.87 and 0.30-0.63. In this data set, the multivariate 4-protein panel made up of TRFE, IC1, ITIH4, and LCAT was optimized for samples from subjects of parity=0. For samples with a parity status of 0, the AUC is 0.77 (shown as solid line). The 4-protein panel was tested for (1) samples from subjects with a parity status of >1 (multiparous) where the AUC is 0.67 (shown as dashed line), and for (2) samples from subjects, regardless of parity status, where the AUC is 0.69 (shown as a dotted line).

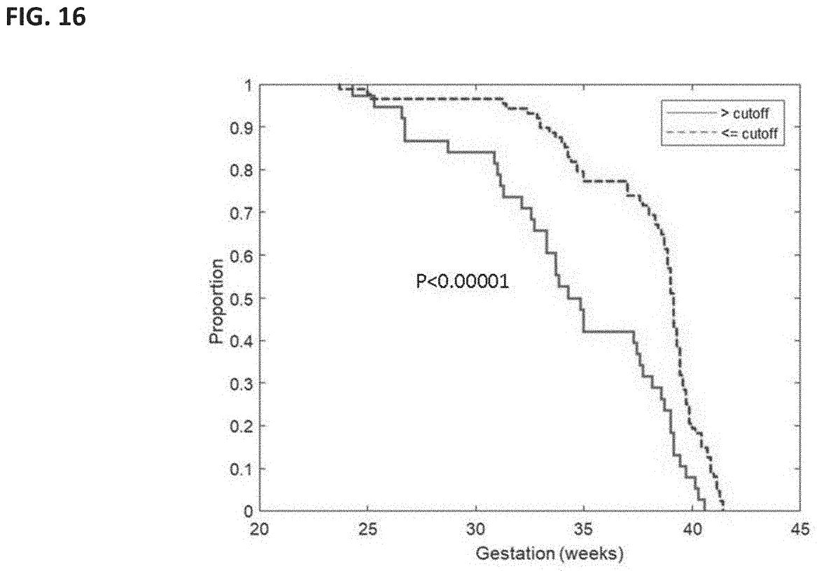

Using the multi-marker panel selected for the primipara (parity=0) mothers, and classifying the pregnancies into high and low risk strata across the test set, FIG. 16 displays the Kaplan-Meier curves for pregnancy survival by week of gestation. The log-rank test indicates that the curves are significantly different (p<0.00001) and demonstrates that a positive marker panel is associated with shorter gestation at all gestational ages, not only those ending <35 weeks.

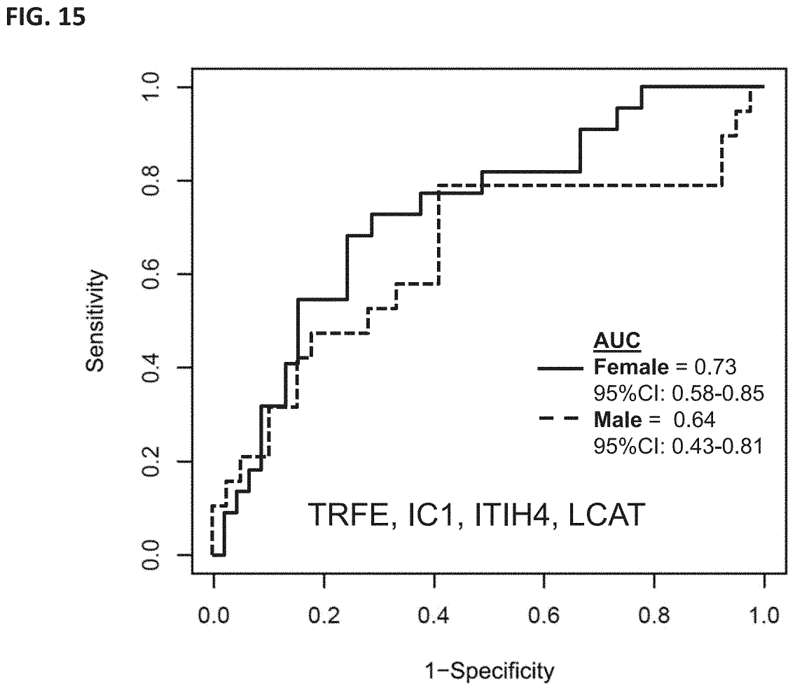

FIG. 15 shows the performance of the same 4 protein panel (TRFE, IC1, ITIH4, and LCAT) by fetal gender. Female fetal gender shows an AUC of 0.73 (95% CI: 0.58-0.85) and male fetal gender shows an AUC of 0.64 (95% IC: 0.43-0.81) Female is shown as a solid line and male is shown as a dashed line.

### Discussion

The 5-plex combination of CMP-associated protein analytes (F13A, FBLN1, IC1, ITIH2 and LCAT) was defined in a training set, with an AUC of 0.74 (95% CI 0.63-0.81) in a testing set. Using a Bayesian logic, given a generalized baseline risk (pre-test probability) of 4.9% for delivery at <35 weeks within the United States, it is expected those that test positive at 10-12 weeks would now have a post-test risk (post-test probability) of 13%, while those with a negative test would be reduced to a 1% risk. It is expected that along with addition of clinical risk scoring based upon maternal characteristics, multi-marker panels could improve these performance metrics.

Additionally, the predictive characteristics of CMP-associated protein analytes to predict SPTB before the end of 35 weeks gestation among nullipara is described. In this population, using a separate set of CMP protein markers, an AUC of 0.77 (95% CI 0.61-0.90) was observed. With a sensitivity of 0.63, this indicates a specificity of 0.86. Again, framed as a Bayesian argument, a pre-test probability of risk of 4.9% for delivery at <35 weeks implies a post-test probability of risk of 20% if positive and 2% if negative. In this population of patients where prior history is lacking, these results imply a potentially clinically useful stratification for the risk of SPTB before the end of 35 weeks.

Table 14A shows peptides that can be detected in the LC-MCS MRM mode to detect the 4 protein panel (TRFE, IC1, ITIH4, and LCAT).

Table 14B shows peptides that can be detected in the LC-MCS MRM mode to detect the 5 protein panel (F13A, FBLN1, IC1, ITIH2, and LCAT).

Table 15A shows the isotope-labeled reference peptides (isotopic standards) used in the LC-MCS MRM mode for detecting the 4 protein panel (TRFE, IC1, ITIH4, and LCAT).

Table 15B shows the isotope-labeled reference peptides (SIS, isotopic standards) used in the LC-MCS MRM mode for detecting the 5 protein panel (F13A, FBLN1, IC1, ITIH2, and LCAT).

There are only limited existing available risk stratification methods available at the end of the first trimester. Such methods amount primarily to an individual's pregnancy history. To date, history has become the most important single metric for gauging a patient's potential for delivery.

With this example, it is demonstrated that CMP-associated protein analytes collected at the end of the first trimester have the ability to be predictive of the risk of birth at <35 weeks gestation.

While the described invention has been described with reference to the specific embodiments thereof it should be understood by those skilled in the art that various changes may be made and equivalents may be substituted without departing from the true spirit and scope of the invention. The various embodiments described above can be combined to provide further embodiments. In addition, many modifications may be made to adopt a particular situation, material, composition of matter, process, process step or steps, to the objective spirit and scope of the described invention. All such modifications are intended to be within the scope of the claims appended hereto.

1-109. (canceled)

110. A computer-implemented method for generating a model to assess a risk of spontaneous preterm birth, the method comprising:
obtaining a dataset, the dataset comprising measurements associated with a plurality of markers derived from each of a plurality of subjects;
implementing a machine learning analysis to associate a set of markers within the plurality of markers with spontaneous preterm birth, wherein implementing the machine learning analysis generates a model to assess the risk of spontaneous preterm birth.

111. The computer-implemented method of claim 110, wherein assessing the risk comprises classifying a subject as being at one of increased risk or decreased risk of spontaneous preterm birth.

112. The computer-implemented method of claim 110, wherein the model executes at least one classification rule to assess the risk of spontaneous preterm birth, and wherein the at least one classification rule comprises at least one of binary decision trees, artificial neural networks, discriminant analyses, logistic classifiers, and support vector classifiers.

113. The computer-implemented method of claim 110, wherein the model executes at least one classification rule to assess the risk of spontaneous preterm birth, wherein the at least one classification rule produces a receiver operating characteristic (ROC) curve, and wherein the ROC curve has an area under the curve (AUC) of at least 0.6.

114. The computer-implemented method of claim 110, wherein the set of markers comprises one or more markers of Table 14A.

115. The computer-implemented method of claim 110, wherein the set of markers comprises one or more markers of Table 14B.

116. A method for stratifying the risk of spontaneous preterm birth in a subject, the method comprising:
determining measurements associated with at least two markers in a sample; and
executing a classification rule based on the measurements,
wherein the execution of the classification rule includes performing a receiver-operating-characteristic (ROC) curve analysis on the measurements, and
wherein the execution of the classification rule stratifies the risk of spontaneous preterm birth in the subject.

117. The method of claim 116, wherein the ROC curve analysis produces a ROC curve, wherein the ROC curve has an area under the curve (AUC) of at least 0.6.

118. The method of claim 117, wherein execution of the classification rule stratifies the subject as being at an increased risk of spontaneous preterm birth.

119. The method of claim 116, wherein the classification rule is configured to have a sensitivity of at least 75%, at least 85%, or at least 95%.

120. The method of claim 116, wherein execution of the classification rule produces a correlation between preterm birth or term birth with a p value of less than at least 0.05, wherein the execution of the classification rule stratifies the subject as being at an increased risk of spontaneous preterm birth.

121. The method of claim 116, wherein the at least two markers are selected from the markers of Table 14A.

122. The method of claim 116, wherein the at least two are selected from the markers of Table 14B.

123. A computer-implemented method for training a machine learning model, the method comprising:
obtaining a dataset, the dataset comprising measurements associated with a plurality of markers derived from each of a plurality of subjects;
performing a receiver-operating-characteristic (ROC) analysis on the dataset, wherein the ROC analysis ranks each marker in the plurality of markers for its ability to distinguish spontaneous preterm birth from term birth;
extracting co-expression patterns among at least two markers in the plurality of markers using a differential dependency network (DDN); and
training a machine learning model using the ROC analysis and the co-expression patterns.

124. The computer-implemented method of claim 123, wherein the machine learning model is a multivariate linear model.

125. The computer-implemented method of claim 123, wherein implementing the machine learning model classifies a subject as belonging to at least one of a first class or a second class, wherein the first class is associated with spontaneous preterm birth and the second class is associated with term birth.

126. The computer-implemented method of claim 123, wherein the machine learning model executes a classification rule to classify a sample as belonging to one of a preterm birth class or a term birth class.

127. The computer-implemented method of claim 126, wherein the at least one classification rule produces a receiver operating characteristic (ROC) curve, and wherein the ROC curve has an area under the curve (AUC) of at least 0.6.

128. The computer-implemented method of claim 123, wherein the machine learning model associates a set of markers within the plurality of markers with spontaneous preterm birth.

129. The computer-implemented method of claim 128, wherein the set of markers comprises one or more markers of Table 14A.

130. The computer-implemented method of claim 128, wherein the set of markers comprises one or more markers of Table 14B.

131. A system to assess risk in a subject, the system comprising:
(a) a processor; and
(b) memory coupled to the processor, the memory to store:
(i) a first dataset comprising a first plurality of measurements associated with a plurality of markers derived from each of a plurality of subjects;
(ii) a second dataset comprising a second plurality of measurements associated with the plurality of markers derived from another subject; and
(iii) computer-readable instructions to:
(1) implement a machine learning analysis to associate a set of markers within the plurality of markers within the first dataset, wherein the machine learning analysis generates a model to assess the risk of spontaneous preterm birth; and
(2) execute a classification rule based on the second plurality of measurements from the other subject, wherein the execution of the classification rule assesses the risk of spontaneous preterm birth in the other subject.

132. A system to assess a risk of spontaneous preterm birth in a subject, the system comprising:
(a) a processor; and
(b) memory coupled to the processor, the memory to store:
(i) a dataset comprising measurements associated with a plurality of markers derived from a subject; and
(iii) computer-readable instructions to execute a classification rule based on the measurements from the subject, wherein the execution of the classification rule assesses the risk of spontaneous preterm birth in the subject.
